(19) United States
(12) Patent Application Publication
(10) Pub. No.: US 2003/0165892 A1
(43) Pub. Date: Sep. 4, 2003

(54) Unigene unidirectional antisense library

(75) Inventors: Jong-Gu Park, Daegu (KR); Ik-Jae Moon, Daegu (KR); Yun-Han Lee, Daegu (KR)

Correspondence Address: SQUIRE, SANDERS & DEMPSEY L.L.P., 14th Floor, 801 S. Figueroa Street, Los Angeles, CA, 90017-5554, US

(73) Assignee: WELGENE PHARMACEUTICALS, INC.

(21) Appl. No.: 10/147,264

(22) Filed: May 15, 2002

(30) Foreign Application Priority Data
May 17, 2001 (KR) 2001-27071

(51) Int. Cl. C12Q 1/70; C12Q 1/68; C07H 21/02; C07H 21/04; C12N 7/00

(52) U.S. Cl. 435/6; 435/5; 435/235.1; 435/325; 536/23.1

(57) ABSTRACT

The present invention provides a high throughput system for functional genomics using a unigene antisense library comprising LC-antisense compounds. The antisense compounds were specific and effective for the elimination of target mRNA. Thus, the system of the present invention is used as temporary knock-down system to unveil functions of genes critical for diseases. The system of the present invention can be adopted not only for functional genomics but also for effectively validating target for antisense or other molecular therapeutics against various malignancies, infections, and other diseases by blocking specific genes involved in the disease.

### BACKGROUND OF THE INVENTION

1. Field of the Invention

The present invention relates to the field of antisense technology. The present invention also relates to using the antisense technology in therapeutics and in gene function identification systems. The present invention relates to a high-throughput system for functional genomics using a unigene antisense library. And in particular, the present invention relates to a system for massively screening genes for their functions.

2. Description of the Background

As most of the genetic information in human genome has been deciphered, many new methods for screening genes and analyzing their functions have been studied and developed at different institutions in the world. These methods have provided new information to understand the biochemical and physiological mechanisms of cell viability and the etiology of diseases. The molecular bases of many incurable diseases will be better understood, and concomitantly more effective therapeutic agents will be developed.

Most human diseases are caused by abnormal gene expression. Genetic causes of disease are manifest in a variety of ways such as termination of gene expression by direct DNA damage, and abnormal transcription and/or translation. Abnormal expression of proto-oncogene expression can cause cancer (Brown et al., Proc. Natl. Acad. Sci. USA, 87(2), 538-542 (1990); Adams et al., Proc. Natl. Acad. Sci. USA, 80(7), 1982-1986 (1983); and Seth et al., Oncogene, 5(12), 1761-1767 (1990)). It was reported that the occurrence and progression of immune-diseases are closely related to the overproduction or underproduction of cytokines (Waszczykowska et al., Mediators Inflamm., 8(2), 93-100 (1999); Pulsatelli et al., J. Rheumatol., 26(9), 1992-2001 (1999). Chronic or incurable diseases, such as various types of cancer, and immune diseases, are caused by abnormal gene expression. Therefore, it is conceivable that these diseases can be controlled by modulation of gene expression.

It is known in the art that one way to control gene expression is by introducing to cells antisense oligos that are complementary to specific mRNA so that the antisense oligonucleotide may bind to the target mRNA and thus eliminate the target mRNA.

Antisense molecules bind to complementary sequences of mRNA through Watson-Crick base pairing. Antisense oligonucleotides (AS-oligos) have been valuable in the functional study of a gene by reducing gene expression in a sequence specific manner (Thompson et al. Nature, 314, 363-366 (1985); Melani et al. Cancer Res., 51, 2897-2901 (1991); Anfossi et al. Proc. Natl. Acad. Sci. USA, 86, 3379-3383 (1989)). Intense effort has also been made to develop antisense anticancer agents that eliminate aberrant expression of genes involved in tumor initiation and progression (Kamano et al. Leuk. Res., 14, 831-839 (1990); Melotti et al. Blood, 87, 2221-2234 (1996); Ferrari et al. Cell growth Differ., 1, 543-548 (1990); Ratajczak et al. Blood, 79, 1956-1961 (1992); Kastan et al. Blood, 74, 1517-1524 (1989); Thaler et al. Proc. Natl. Acad. Sci. USA, 93, 1352-1356 (1996); Wagner Nature, 372, 333-335 (1994)). Synthetic AS-oligos have been widely utilized for their ease of design and synthesis as well as potential specificity to a target gene. Antisense inhibition of gene expression is believed to be achieved either through RNaseH activity following the formation of antisense DNA-mRNA duplex or through steric hindrance of the mRNA movement to bind a ribosomal complex (Dolnick Cancer Invest., 9, 185-194 (1991)). There has also been an effort to inhibit gene expression by employing oligonucleotides that form triple helices aimed at the promoter region of the genomic DNA. Moreover, duplexed oligonucleotide decoys that compete with the promoter region of genomic DNA has also been formed (Young et al. Proc. Natl. Acad. Sci. USA, 88, 10023-10026 (1991)). Efficacy of AS-oligos has been validated in animal models as well as in several recent clinical studies (Offensperger et al. EMBO J., 12, 1257-1262 (1993); Tomita et al. Hypertension, 26, 131-136 (1995); Nesterova et al. Nat. Med., 1, 528-533 (1995); Roush Science, 276, 1192-1193 (1997)). In addition, the first antisense drug was approved for CMV retinitis in US and Europe.

Expectations for AS-oligos have, however, frequently met with disappointment as results have not always been unambiguous. Some of the problems of using AS-oligos have been inaccessibility to a target site (Flanagan et al. Mol. Cell Biochem., 172, 213-225 (1997); Matsuda et al. Mol. Biol. Cell, 7, 1095-1106 (1996)), instability to nucleases (Akhtar et al. Life Sci., 49, 1793-1801 (1991); Wagner et al. Science, 260, 1510-1513 (1993); Gryaznov et al. Nucleic Acids Res., 24, 1508-1514 (1996)), lack of sequence specificity, and various negative side effects in vivo. The stability of AS-oligos has improved to a certain extent by using chemically modified oligos, which are the so-called second generation AS-oligos (Helene Eur J Cancer, 27(11),1466-71 (1991); Bayever et al. Antisense Res. Dev. 3(4), 383-90 (1993); Baker et al. Biochim. Biophys. Acta., 1489, 3-18 (1999)). Phosphorothioate (PS)- and methylphosphonate (MP)-oligos, have been exhaustively studied and are utilized mainly to augment stability to nucleases. However, each of the modified AS-oligos exhibit both lack of sequence specificity and insensitivity to RNaseH. Further, there has been concern over inadvertent introduction of mutations during DNA replication or repair caused by recycling of hydrolyzed modified nucleotides.

A series of distinct antisense molecules with enhanced stability, the so-called ‘third generation AS-oligos’, having 1) a stem-loop structure, 2) the CMAS (Covalently-closed Multiple Antisense) structure and 3) the RiAS (Ribbon Antisense) structure (Moon et al. Biochem J., 346, 295-303 (2000); Matsuda et al. Mol. Biol. Cell, 7, 1095-1106 (1996); Moon et al. J Biol Chem., 275(7), 4647-53 (2000)) have been described. Both CMAS and RiAS-oligonucleotides exhibit enhanced stability to exonucleases and nucleases in biologic fluids. These antisense molecules are also efficacious in the specific reduction of target mRNA. However, there is a need in the art to develop an antisense molecule with greater facility and enhanced binding efficiency.

Certain bacteriophages, such as M13 bacteriophage, have a single-stranded circular genome, which has been employed for DNA sequencing analyses as well as mutagenesis studies. M13 phagemid, which is a plasmid used in the construction of a recombinant bacteriophage, can be engineered to produce a large quantity of circular single-stranded genomic. DNA that contains an antisense sequence to a specific gene. This approach for producing antisense DNA takes advantage of the stability to exonucleases associated with the covalently closed structure, high sequence fidelity, elimination of laborious target site search and easy construction of an antisense library.

Synthetic AS-oligos are about 15 to 25 bases long, and bind only to a single target site and eliminate substrate mRNA. However, most chronic and end stage human diseases show multiple genetic disorders. Thus, antisense molecules that can target multiple genes would appear to be more effective in treating such diseases. In order to satisfy such a need, it would be attractive to devise an antisense molecular system with multiple targeting ability. However, synthesizing such molecules would not be practical because of the difficulty of chemically synthesizing them.

AS-oligos have a fundamental and inherent drawback for use in functional genomics. First, chemically modified AS-oligos cause nonspecific binding to target mRNA and as a result, they are less effective and are often cytotoxic to cells, which of course creates false positive results. Second, synthetic AS-oligos, due to their short size (usually 15 to 25 bases) may not be uniformly effective in binding to their targets because they require a target search before effectively binding to their target mRNA. Third, there is a possibility that an error in synthesis of AS-oligos decreases the specificity of their binding. Fourth, production cost for AS-oligos is high. And finally, when AS-oligos are used in functional genomics, these AS-oligos sometimes show incomplete antisense activity against their target mRNAs, thus generating unreliable and ambiguous data.

Current functional genomics systems using DNA chip technology, proteomics and so on are limited to providing gene expression profiles. However, to perform definitive functional analysis of genes, additional assays are required to be performed downstream of a particular gene inactivation.

Thus, there is a need in the art for a gene functionalization system to determine the functions of yet uncharacterized genes.

### SUMMARY OF THE INVENTION

The claimed invention overcomes the above-mentioned problems, and provides antisense molecules, compositions of antisense molecules, a method of making the antisense molecules, and a method of using the claimed molecules and compositions which provide the advantage of inhibiting or significantly modifying the expression of certain targeted genes. In the case that expression of these targeted gene(s) is responsible for causing cancer, then administering the inventive antisense molecules to the cells results in the ablation of the target RNA, which will inhibit proliferation of the cells, which in turn will result in curing or at least improving the survival associated with the cancer.

Applicants have developed large circular nucleic acid molecules that contain at least one target-specific antisense region by using a phagemid vector having a single-stranded circular genome. This large circular nucleic acid molecule may be called an LC-antisense compound. In a particular embodiment of the invention, applicants have constructed a phage genomic antisense library comprising separately individually cloned sequence verified cDNA. The antisense library allows screening and analysis of the functions of genes with speed and accuracy. Thus, high throughput and massive functional genomics systems are provided. Furthermore, the present invention may be used for validating therapeutic antisense compounds for chronic or incurable diseases.

In one aspect, the present invention is directed to massive functional genomics using LC-antisense compounds. LC-antisense compounds provide an effective platform for functionalization of a large number of genes with unknown functions and of genes with known but additional unknown functions. In addition, the present invention also may be used for the direct development of antisense molecular therapeutics.

LC-antisense compounds show superior and sensitive antisense activity even with small doses as compared with conventional AS-oligos. Typically, LC-antisense compounds are derived from cloned cDNAs in a phagemid vector, and large, single-stranded DNAs with target-specific antisense regions are generated. Thus, due to the large size of the molecule, LC-antisense compounds do not require a target site search for effective antisense activity and stability to degradation by nucleases. In addition, antisense libraries can be constructed relatively easily by introducing tens of thousands of different genes or gene fragments into phagemid vectors all at once. Large-scale generation of bacterially produced LC-antisense compounds can be easily obtained at low cost. Finally, the bacteriophage genomic antisense compound as applied to the area of massive functional genomics provides speed, low cost, and analytical accuracy.

It is also to be understood that as the LC-antisense compounds are used therapeutically, the invention is not limited to treating cancer. The principles of the antisense compound of the invention may be applied to efficiently ablate any target RNA. Any phenotypic manifestation of this chemical activity in the form of cancer treatment, eliminating adverse effects of viral infection, treating metabolic diseases, immunologic disorders, and so on may be the result of antisense molecular therapy.

The LC-antisense compounds chosen from a large antisense library may be adapted to configure an antisense array system such as a macroarray or microarray system. The antisense array system may be effectively utilized for functional comparison of the antisense compounds among different types of cells treated with the antisense compounds. Comparative functional diagnostics as well as understanding the underlying molecular mechanism of a disease may be performed by employing the antisense array assembly system of the invention.

A panel of antisense compounds used in the antisense array assembly may be chosen based on the results obtained from either a primary functional assay using an antisense library or from conventional expression profiling or expression tracking system, such as DNA chip, SAGE, Toga and proteomics.

The invention further includes compositions of the claimed antisense molecules together with a pharmaceutically acceptable carrier.

The present application is directed to a library of a multitude of unique single-stranded nucleic acids, said library comprising:

a multiplicity of compartments, each of said compartments comprising one or more single-stranded LC-antisense compound derived from recombinant bacteriophage or phagemid vector comprising at least one unique unidirectional antisense nucleic acid insert in an aqueous medium,

wherein said LC-antisense compound is capable of being introduced into a host cell, and which is capable of specifically binding to a nucleic acid in said host cell that is substantially complementary to said unique antisense nucleic acid insert.

In the above described library, the specificity of the unique antisense nucleic acid insert to a target gene may be known or unknown at the time said library is first made. Furthermore, in the library, the specificity of a target host cell nucleic acid that controls the expression of a phenotype of the host cell may be known or unknown at the time the library is first made. And the host cell may be a prokaryotic or eucaryotic cell. Preferably, the cell is eucaryotic. Furthermore, the library may be placed in compartments that contain from about 0.1 μM to about 1 μM of said LC-antisense compound per ml of aqueous medium. Moreover, the bacteriophage or phagemid vector may be derived from a filamentous bacteriophage. And the filamentous bacteriophage may be an M13 bacteriophage. Furthermore, the bacteriophage or phagemid vector may comprise bacteriophage or phagemid genomic sequence in which is inserted said unique antisense nucleic acid insert sequence.

In the library, the bacteriophage or phagemid vector may comprise more than one kind of unique antisense nucleic acid insert sequence. And the multiplicity of compartments may comprise a multi-well format of at least 6 wells, and preferably 96 wells. Furthermore, the library may be configured to be made and used in a substantially automated process. And the host cell may be abnormal such that modulation of gene expression is beneficial in returning said host cell to its normal state.

In the library, the abnormality of the host cell may be caused by a variety of agents, such as but not limited to cancer, viral infection, immunologic disorders or metabolic diseases. Cancer may include, but not limited to, liver cancer, lung cancer, stomach cancer, colon cancer, leukemia, cervical cancer, prostate cancer, rectal cancer, bladder cancer, pancreatic cancer, skin cancer, ovarian cancer, kidney cancer or breast cancer. Viral infection may be caused by a virus that includes, but is not limited to, human papilloma virus (HPV), HIV, small pox, mononucleosis (Epstein-Barr virus), hepatitis, or respiratory syncytial virus (RSV). Metabolic disease may include, but not limited to, phenylketonuria (PKU), primary hypothyroidism, galactosemia, abnormal hemoglobins, types I and II diabetes, or obesity. Immunological disorder may include, but not limited to, Sjogren's Syndrome, antiphospholipid syndrome, immune complex diseases, Purpura, Schoenlein-Henoch, immunologic deficiency syndromes, systemic lupus erythematosus, immunodeficiency, rheumatism, kidney, or liver sclerosis.

The present invention is also directed to a method of making a library comprising a multitude of unique single-stranded nucleic acids, which comprises one or more single-stranded LC-antisense compound derived from recombinant bacteriophage or phagemid vector comprising at least one unique unidirectional antisense nucleic acid insert, comprising:

inserting a nucleic acid fragment unidirectionally into said bacteriophage or phagemid vector by unidirectionally cloning the nucleic acid fragments into said vector.

The method may further comprise preparing bacterial transformants by introducing the vector containing the insert into competent bacterial cells to make bacterial transformants; and then infecting said transformants with helper phage to produce said single-stranded nucleic acid library.

In another aspect of the invention, the invention is directed to a method for specifically inhibiting growth of liver cancer cells, comprising administering to said cells a large circular antisense compound targeted to polymyositis/scleroderma autoantigen, ESTs (N21972), Nuclear matrix protein p84, Gamma-aminobutyric acid (GABA) A receptor beta 3, SRY (sex-determining region Y)-box 9, ESTs (H13112), ESTs (AW294133), Primase, polypeptide 1 (49 kD), Human EV12 protein gene, epidermal growth factor receptor pathway substrate 8 or protein tyrosine phosphatase, non-receptor type 2.

In yet another aspect of the invention, the invention is directed to a method for specifically inhibiting growth of lung cancer cells, comprising administering to said cells a large circular antisense compound targeted to TGF-β stimulated protein, TSC-22, General transcription factor II H, Cytochrome P450, subfamily IIIA, polypeptide 7, KIAA0094 protein (D42084), MAX dimerization protein, Serine/treonine kinase 13 (aurora/IPL 1-kike), ESTs (AIO57094), Ras-related GTP-binding protein, MHC class I region ORF, or Tumor necrosis factor receptor superfamily, member 7.

In yet another embodiment of the invention, the invention is directed to a high throughput system for conducting a functional genomics assay with a unigene unidirectional antisense library comprising the steps of:

(i) forming large circular antisense molecule-carrier complexes with said unigene unidirectional antisense library;

(ii) transfecting the complexes into host cells to eliminate endogenously expressed substantially complementary transcripts;

(iii) screening for a change in phenotype of the host cell;

(iv) identifying the gene that caused the change in phenotype in (iii).

The high throughput system may require further functional testing. And the high throughput system may further comprise comparing the gene sequence obtained in step (iv) with previously verified clone information to determine homologous genes or the full gene sequence.

In the high throughput system, the carrier that is used may include, but not limited to, liposomes, cationic polymers, HVJ-liposomes complexes, peptides or viruses. And the large circular antisense molecule and carrier may be mixed in a desirably optimum ratio that may be determined using routine experimentation, and which may typically comprise about 1:3 or about 1:4 w/w.

The assayed phenotype may be any that is desired, and may include without limitation cell morphology, cell proliferation, cell apoptosis, or cell reaction to a substrate.

An assay that may be used include, but not limited to, RT-PCR, Western blot analysis, immunoassay, MTT reduction assay, [³H]-thymidine incorporation assay, colony formation assay, DNA synthesis and chromatin activation, analysis of apoptosis by inspection of cell morphological changes, chromosomal condensation or fragmentation, DNA fragmentation, quantitative assay for apoptosis, signaling mechanisms of apoptosis, activation of cell cycle regulators, complex formation between cell cycle regulators, or assays for changes of metabolic, morphological, physiological and biochemical phenotypes in vitro and in vivo.

The invention is also directed to a high throughput system for conducting massive functional genomics assays, which is performed by applying a unigene unidirectional antisense library to a cell line of a particular disease comprising the following steps:

1) making an antisense library by massively parallel production of LC-antisense compounds to a large number unigenes;

2) plating a population of host cells in multi-well plates;

3) forming an LC-antisense compound-carrier complex with the antisense library of step 1);

4) performing primary gene functional analysis by transfection of the complex of step 3) into the population of host cells; and

5) performing additional functional analysis of the gene screened in step 4).

In this high throughput system, the unigene LC-antisense compound may be prepared by the steps of:

1) preparing a cDNA fragment of a target gene;

2) preparing a recombinant phage or phagemid by inserting the cDNA fragment of step 1) into a phage or phagemid vector that is capable of producing LC-antisense compounds; and

3) producing the LC-antisense compounds containing antisense sequence of the unigene as a part of a single-stranded circular genome made by the recombinant phage or phagemid of step 2).

In still another embodiment of the invention, the invention is directed to a high throughput system for massive functional genomics performed by applying a macroarray or microarray assembly to various kinds of disease cells comprising the steps of:

1) making an antisense array by selecting unigene LC-antisense compounds;

2) plating a population of host cells in multi-well plates;

3) forming LC-antisense compound-carrier complexes on the antisense array of step 1);

4) performing primary gene functional analysis by transfection of the complexes of step 3) into the population of cells; and

5) performing additional functional assays of the genes screened in step 4).

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows a schematic diagram for generating rat TNFα-M13 antisense molecule (TNFα-M13AS).

FIG. 2 shows sequence analysis of TNFα-M13AS confirming the antisense nature of the TNF-α insert.

FIG. 3A shows the results of treating TNFα-M13AS with various enzymes. TNFα-M13AS was confirmed to be single-stranded. Lane 1: Plasmid DNA containing TNFα-cDNA (TNFα-plasmid); Lane 2: TNFα-M13AS; Lane 3: TNFα-plasmid digested with Xho I; Lane 4: TNFα-M13AS digested with Xho I; Lane 5: TNFα-plasmid digested with S1 nuclease; Lane 6: TNFα-M13AS digested with S1 nuclease; Lane 7: TNFα-plasmid digested with Xho I and exonuclease III; and Lane 8: TNFα-M13AS digested with Xho I and exonuclease III.

FIG. 3B shows the stability of TNFα-M13AS to nucleases. Lane 1: TNFα-plasmid; Lane 2: TNFα-M13AS; Lane 3: TNFα-plasmid+FBS; Lane 4: TNFα-plasmid digested with Xho I+FBS; Lane 5: TNFα-M13AS+FBS; Lane 6: TNFα-M13AS and liposome complex+FBS; Lane 7: TNFα-plasmid+calf serum; Lane 8: TNFα-plasmid digested with Xho I+calf serum; Lane 9: TNFα-M13AS+calf serum; and Lane 10: TNFα-M13AS and liposome complex+calf serum.

FIG. 4A shows the results of RT-PCR using a TNFα-specific primer pair and a β-actin specific primer pair. Rat TNF-α expression was specifically inhibited by TNFα-M13AS of the present invention. Lane 1: Liposome; Lane 2: TNFα-M13AS; Lane 3: TNFα-M13 sense; and Lane 4: Single-stranded phage genomic DNA without rat TNF-α cDNA.

FIG. 4B shows the results of amplifying IL-1β and GAPDH transcripts by RT-PCR, confirming that TNFα-M13AS specifically inhibits the expression of rat TNF-α. Lane 1: Liposome; Lane 2: TNFα-M13AS; Lane 3: TNFα-M13 sense; and Lane 4: Single-stranded phage genomic DNA without rat TNF-α cDNA.

FIG. 4C shows Southern blot data using rat TNF-α specific hybridization probe, confirming that TNFα-M13AS specifically inhibits the expression of rat TNF-α. Lane 1: Liposome; Lane 2: TNFα-M13AS; Lane 3: TNFα-M13 sense; and Lane 4: Single-stranded phage genomic DNA without rat TNFα-cDNA.

FIG. 5 shows ELISA assay data that measure the quantity of rat TNF-α protein secreted from cells. The data show that rat TNF-α protein expression decreases in response to administration of TNFα-M13AS. Lane 1: Liposome; Lane 2: TNFα-M13AS; and Lane 3: TNFα-M13SS.

FIG. 6A shows RT-PCR results confirming that endogenous NF-κB expression decreases in response to administration of NFκB-M13AS.

FIG. 6B shows Southern blot results using an NF-κB specific probe. Data confirm that human NF-κB expression decreases in response to administration of NFκB-M13AS.

FIG. 7 shows a schematic diagram of a high-throughput system for functional genomics applying the unigene antisense library to cells of a particular disease.

FIG. 8 shows a schematic diagram of a high-throughput system for functional genomics applying a macroarray assembly comprising large circular antisense compounds (LC-antisense compounds) selected from a unigene antisense library to various cell types.

FIG. 9 shows a schematic diagram of gene functionalization using the unigene antisense library.

FIG. 10 shows transformants grown on selection media containing ampicillin. Prior to the production of LC-antisense compounds, recombinant pBluescript SK(−) phagemids with unigene cDNA inserts were introduced into competent bacterial cells by the calcium chloride method.

FIG. 11 shows examples of massive purification of recombinant phagemids from bacterial transformants.

FIG. 12 shows confirmation of insert length. Purified recombinant phagemids were digested with restriction enzymes used in subcloning the unigene cDNA, and electrophoresed on a 1% agarose gel.

FIG. 13 shows rapid construction of a unigene antisense library comprising LC-antisense compounds.

FIGS. 14A and 14B show massive gene functionalization using the unigene antisense library. Liver cancer cell line (HepG2) was transfected with LC-antisense compound-carrier complexes in 96-well plates. MTT reduction assay was performed to observe changes in cell proliferation. FIG. 14A shows 96-well plates containing MTT reagents. FIG. 14B shows results of screening for genes related to liver cancer by percentage calculation of growth inhibition of cells treated with LC-antisense compounds.

FIGS. 15A and 15B show massive gene functionalization using the unigene antisense library. Lung cancer cell line (NCI-H1299) was transfected with LC-antisense compound-carrier complexes in 96-well plates. MTT reduction assay was performed to observe changes in cell proliferation. FIG. 15A shows 96- well plates containing MTT reagents. FIG. 15B shows results of screening for genes related to liver cancer by percentage calculation of growth inhibition of cells treated with LC-antisense compounds.

FIGS. 16A-16D show examples of gene functionalization performed with a clone from the unigene antisense library on the HepG2 cell line. Measurements of cell growth of HepG2 were performed by light microscopy (FIGS. 16A and 16C), MTT assay (FIG. 16B) and [³ H]-thymidine incorporation assay (FIG. 16D).

FIGS. 17A-17D show examples of gene functionalization performed with a clone from the unigene antisense library on the HepG2 cell line. Measurements of cell growth of HepG2 were performed by light microscopy (FIGS. 17A and 17C), MTT assay (FIG. 17B) and [³H]-thymidine incorporation assay (FIG. 17D).

FIGS. 18A-18D show examples of gene functionalization performed with a clone from the unigene antisense library on the NCI-H1299 cell line. Measurements of cell growth of NCI-H1299 were performed by light microscopy (FIGS. 18A and 18C), MTT assay (FIG. 18B) and [³H]-thymidine incorporation assay (FIG. 18D).

FIGS. 19A-19D show examples of gene functionalization performed with a clone from the unigene antisense library on NCI-H1299 cell line. Measurements of cell growth of NCI-H1299 were performed by light microscopy (FIGS. 19A and 19C), MTT assay (FIG. 19B) and [³H]-thymidine incorporation assay (FIG. 19D).

FIG. 20 shows an example of antisense activity profile of an LC-antisense compound to various kinds of cancer cells. The LC-antisense compound was transfected into different cancer cell lines, Hep3B (liver cancer), NCI-H1299 (non-small lung cancer), AGS (stomach cancer) and HT-29 (colon cancer). Cell growth was measured using MTT assay on a macroarray assembly, and data were compared.

### DETAILED DESCRIPTION OF THE INVENTION

The present invention is based on the discovery that a large circular phage genomic molecule that includes a target specific antisense region, is useful as an effective ablator of gene expression, and as such can be used to determine the function of the target gene. The inventive system can be used in a high throughput manner in a massive functional genomics protocol to determine genes involved in various cellular physiological processes.

It is understood that while the application describes the use of the unigene library for functional genomics assay relating to certain human disease states, the unigene library may be obtained from and used in any organism, so long as it is desired to discover the gene controlling a particular phenotype. Such organisms may encompass bacterial, fungal, plant or animal cells, and the phenotype that is desired to be assayed may be that which is known or yet to be discovered.

In particular, the present invention provides LC-antisense compounds derived from recombinant bacteriophage genome and methods for preparing them. The present invention also provides a unigene antisense library, which may be constructed using bacteriophage genome antisense vectors. Additionally, the present invention provides a high-throughput system for functional genomics using the antisense library.

In the present application, “a” and “an” are used to refer to both single and a plurality of objects.

As used herein, the term “antisense” means antisense nucleic acid (DNA or RNA) and analogs thereof and refers to a range of chemical species having a range of nucleotide base sequences that recognize polynucleotide target sequences or sequence portions through hydrogen bonding interactions with the nucleotide bases of the target sequences. The target sequences may be single- or double-stranded RNA, or single- or double-stranded DNA.

Such RNA or DNA analogs comprise but are not limited to 2′-O-alkyl sugar modifications, methylphosphonate, phosphorothioate, phosphorodithioate, formacetal, 3′-thioformacetal, sulfone, sulfamate, and nitroxide backbone modifications, amides, and analogs wherein the base moieties have been modified. In addition, analogs of molecules may be polymers in which the sugar moiety has been modified or replaced by another suitable moiety, resulting in polymers which include, but are not limited to, morpholino analogs and peptide nucleic acid (PNA) analogs. Such analogs include various combinations of the above-mentioned modifications involving linkage groups and/or structural modifications of the sugar or base for the purpose of improving RNaseH-mediated destruction of the targeted RNA, binding affinity, nuclease resistance, and or target specificity.

As used herein, “antisense therapy” is a generic term, which includes specific binding of large circular antisense molecules that include an antisense segment for a target gene to inactivate undesirable target DNA or RNA sequences in vitro or in vivo.

As used herein, “cell proliferation” refers to cell division. The term “growth,” as used herein, encompasses both increased cell numbers due to faster cell division and due to slower rates of apoptosis, i.e. cell death. Uncontrolled cell proliferation is a marker for a cancerous or abnormal cell type. Normal, non-cancerous cells divide regularly, at a frequency characteristic for the particular type of cell. When a cell has been transformed into a cancerous state, the cell divides and proliferates uncontrollably. Inhibition of proliferation or growth modulates the uncontrolled division of the cell.

As used herein, “chimeric large circular antisense molecule” refers to a large circular nucleic acid molecule comprising a plurality of antisense nucleotide segments that are substantially complementary to a plurality of target genes. The segments of antisense nucleotides may be connected or linked to each other directly or indirectly by use of spacers between each segment.

As used herein, “compartment or compartments” refers to a physical delineation of each member clone of the LC-antisense molecule library. Physical delineation may be in the form of wells such as in multiwell plates. Commonly used are 96 well plates or 96 deep well plates. Another physical barrier may be air, such as by individual spotting on a flat sheet or membrane. It is understood that by compartmentalization it is meant that the clone members are separated from each other. Other barriers may be by encapsulation of individual clones in a membranous material, and the like.

As used herein, “filamentous phage” is a vehicle for producing the large circular antisense molecule of the invention. Phages or phagemids may be used. In this instance, the desired sequence is inserted or cloned into the vehicle so that when a single strand is generated by the phage or phagemid, the large circular antisense molecule is generated. DNA or RNA bacteriophage may be used for this purpose. In particular, filamentous bacteriophage may be used. Filamentous phages such as M13, fd, and f1 have a filamentous capsid with a circular ssDNA molecule. Their life-cycle involves a dsDNA intermediate replicative form within the cell which is converted to a ssDNA molecule prior to encapsidation. This conversion provides a means to prepare ssDNA. The bacteriophage M13 has been adapted for use as a cloning vector.

Phagemid vectors also have filamentous phage f1 Ori region. pBluescript (Stratagene, USA), pGEM-f (Promega, USA), M13mp, pCR2.1, pGL2, pβgal and pSPORT vector and their derivatives may be used. Preferentially, the phagemid vector of M13 bacteriophage, pBluescript SK(−) or KS(−), may be used. One advantage of using a recombinant viral vector based on M13 bacteriophage is that the vector can accomodate a variety of sizes of antisense inserts. Because pBluescript SK(−) phagemid vector has f1(−) origin, the entire nucleotide sequence comprising the antisense form of the target nucleotide sequence and vector originated genes, for example, the ampicillin resistance gene and the lacZ gene, are expressed in single-stranded form.

Another bacteriophage having single-stranded circular genome and having an icosahedral shape is ΦX174. However, this cloning vector has a limitation on the insert size.

As used herein, “functional genomics” or “massive functional genomics” refers to the scientific discipline and utility in biotechnology in which the functions of genes are experimentally determined and identified. If this process is performed with rapidity, in parallel, and in great quantities, it may be termed “high throughput” or “massive” functional genomics.

As used herein, a “gene” refers either to the complete nucleotide sequence of the gene, or to a sequence portion of the gene.

As used herein, the terms “inhibiting” and “reducing” are used interchangeably to indicate lowering of gene expression or cell proliferation or any other phenotypic characteristic.

As used herein, “large circular antisense molecule (LC-antisense molecule)” also referred to as “phage genomic antisense molecule”, or sometimes “large circular nucleic acid molecule”, is a single-stranded molecule, which includes at least one antisense region that is substantially complementary to and binds a target gene or RNA sequence, which inhibits or reduces expression of the gene as well as, in some instances, its isoforms. The circular single-stranded nucleic acid molecule may contain either sense or antisense sequence for one or several genes, so long as the sequence for the target gene is in the antisense form.

Large circular nucleic acid molecule may be synthesized by chemical methods. Typically, it is produced from a filamentous phage system, which includes M13 and phagemids that are derived from it. When the large circular nucleic acid molecule is generated from a phage, it may also be referred to as a “phage genomic antisense compound”.

In one sense, the large circular nucleic acid molecule is longer than a typical oligonucleotide sequence that may be about 20 to 30 nucleotides long. In contrast, the large circular nucleic acid molecule may be at least 3,000 nucleotides long. Typically, the range may be from about 3,000 to about 8,000 nucleotides long. Although a length of about 3,100 to about 7,000 nucleotides may be also useful in the invention, preferred length range may be from about 3,300 to about 6,000 bases.

Alternatively, it is understood that there does not have to be an absolute upper or lower limit to the length of the large circular nucleic acid molecule. This is especially so when a phage is used to generate the large circular nucleic acid molecule, in which case the size of the phage and the size of the insert that encodes at least a portion of the target gene may control the length of the single stranded nucleic acid generated. Thus, in one embodiment, the nucleic acid molecule may be as long as a phage such as a filamentous phage may accommodate.

The large circular nucleic acid molecule may contain both the specific antisense sequence as well as extraneous sequence. Extraneous sequence may include sense or antisense forms of various other genes. Or, if a phage is used to generate the nucleic acid molecule, the extraneous sequence may be the vector sequence. The length of the target specific antisense region of the large circular nucleic acid molecule may be without limitation from a bit lower than about 50 nucleotides to over about 5,000 bases. Typically, the range may be from about 200 to about 3,000 nucleotides. In particular, the range may be from about 400 to about 2,000. The target specific antisense region may be also complementary to an entire gene.

In another embodiment, the antisense molecule may be generated from the genome of a bacteriophage as part of the natural life cycle of the phage.

As used herein, “macroarray” or “microarray” refers to a selected set of LC-antisense compounds, which can be employed to examine the functional profile of the antisense molecules in different types of cells or cell lines.

As used herein, a “gene” refers either to the complete nucleotide sequence of the gene, or to a sequence portion of the gene.

As used herein, “substantially complementary” means an antisense sequence having at least about 70% homology, or preferably, about 80% homology with an antisense compound which itself is complementary to and specifically binds to the target RNA. As a general matter, absolute complementarity may not be required. Any antisense molecule having sufficient complementarity to form a stable duplex or triplex with the target nucleic acid is considered to be suitable. Stable duplex formation depends on the sequence and length of the hybridizing antisense molecule and the degree of complementarity between the antisense molecule and the target sequence.

As used herein, “target” or “targeting” refers to a particular individual gene for which an antisense molecule is made. In an embodiment of the invention, the antisense molecule is made from an insert in a LC-antisense compound. In certain contexts, “targeting” means binding or causing to be bound the antisense molecule to the endogenously expressed transcript so that target gene expression is eliminated. The target nucleotide sequence may be selected from genes involved in various malignancies, including genes involved in the initiation and progression of various diseases such as immune diseases, infectious diseases, metabolic diseases and hereditary diseases or any other disease caused by abnormal expression of genes.

As used herein, “unigene” antisense library refers to a collection of sequence-verified nucleic acid fragments that are optionally inserted into an antisense nucleic acid-generating vector.

Large Circular (LC) Antisense Compounds

The present invention provides LC-antisense compounds having enhanced stability to nucleases and specific activity. The invention is also directed to a method for producing the LC-antisense compounds by using recombinant bacteriophages having a single-stranded circular genome. Further, in one embodiment of the invention, by employing the phage genomic antisense method of the invention, the efficiency of the system as used in massive functional genomics is superior by several hundred fold to that of conventional AS-oligo method. Moreover, contrary to using other indirect systems, such as DNA chip, Serial Analysis of Gene Expression (SAGE), and TIGR Orthologous Gene Alignment (TOGA) database proteomics, massive functional genomics employing the inventive phage genomic antisense system employs a direct gene functionalization system.

The LC-antisense compounds of the present invention may be made by 1) preparing a cDNA fragment having a target nucleotide sequence; 2) preparing a recombinant phage by cloning the cDNA fragment into the phagemid vector that is capable of producing the LC-antisense compound; and 3) producing the single-stranded circular phage genome containing the target antisense sequence in a large scale manner.

It is understood that the LC-antisense compounds may comprise either fragments of a target sequence or the entire gene sequence. Also, it is contemplated that several antisense sequences for a plurality of different genes may be inserted into one single-stranded phage genome.

LC-antisense compounds have strong replication fidelity because the compound is replicated by DNA polymerase in bacterial cells. Since DNA polymerase has proof reading capabilities, the fidelity of LC-antisense compound is greater than chemically synthesized AS-oligos. Moreover, LC-antisense compounds of the present invention are cheaper to make than the chemically synthesized oligonucleotides. High cost required for the synthesis of high quality AS-oligos has been regarded as an obstacle for preclinical and clinical trials.

LC-antisense compounds are stable against degradation by nucleases, and are target specific. In contrast, when chemically modified oligonucleotides are introduced to the cells, mutations as well as retardation of blood clotting or complement activation reaction are induced. Additionally, when the chemically synthesized oligonucleotides are eventually degraded, the individual nucleotides are recycled back into the genomic DNA through DNA replication or repair mechanisms. Incorporation of the chemically modified nucleotides into genomic DNA will likely cause mutations.

Without being bound by any particular theory regarding why the LC-antisense compounds have these advantageous properties, it is believed that when a large target-specific antisense sequence such as the LC-antisense compound of the invention is used, searching for an open site along the target mRNA is likely to be easily achieved.

In exemplified embodiments of the invention, LC-antisense compounds against TNF-α and NF-κB were prepared. Each of these LC-antisense compounds was about 3.7 kb in size and was stable to nuclease degradation. The TNF-α specific insert was 708 bp, and was effective in ablating TNF-α gene expression. The NF-κB specific insert was 700 bp, and it too was effective in ablating NF-κB gene expression. This presents a significant advantage over using chemically synthesized oligonucleotides, which require a careful and laborious process of determining the effective target sites. Thus, the LC-antisense compound is facile to use and saves time and effort associated with searching for effective target sites.

In addition, the efficiency of the liposome mediated delivery of LC-antisense compounds is close to that of a plasmid because of its sufficiently long sequence, which contributes to its excellent antisense activity. The rate of cellular uptake of LC-antisense compound-liposome complex was better than the rate of uptake of oligonucleotides alone.

LC-antisense compound generally includes the antisense sequence and either antisense or sense form of the nucleotide sequences of the vector encoded genes such as ampicillin resistance gene and β-galactosidase gene (lacZ). However, LC-antisense compounds did not cause any significant amount of non-specific inhibition of gene expression. In contrast, chemically modified synthetic oligonucleotides cause significant problems associated with non-specific inhibition.

Regarding the size of the antisense molecule, conventional wisdom in the field of antisense research has discouraged using long antisense molecules because it was thought that longer AS-oligos tend to be less specific, harder to synthesize and inefficient in cellular uptake. Indeed, chemically modified second generation AS-oligos such as phosphorothioate modified oligos lose sequence specificity as the length of the AS-oligos is extended. Furthermore, synthesis of linear AS-oligos becomes increasingly difficult as the oligonucleotides are extended to longer sequences, and sequence fidelity declines markedly as the length of the AS-oligos increases. However, in contravention of this teaching, applicants have discovered that antisense activity is dependent on the length of the antisense sequence. If the length of the antisense sequence is decreased, the antisense activity also decreases. Thus, LC-antisense compounds exhibit sequence specificity, resistance to nuclease degradation, and non-toxicity.

Additional advantages associated with the phage genomic antisense molecule is the broad tolerance in sequence variation. The genomic antisense molecule of the invention may be effective as long as patches of identical sequences with more than about 15 consecutive nucleotides are conserved between different gene variants. This property is particularly useful in targeting polymorphic strains of pathogenic viruses such as HIV and HBV in which the same antisense molecule may be used against the variant forms. In addition, this type of phage genomic antisense molecule generated from one species such as humans may be used to study gene function in other species such as rodents, as long as the sequence divergene between the source and target organism is not spread evenly along the coding sequence.

Some of the significantly advantageous features of LC-antisense compounds are as follows:

1. LC-antisense compounds have an improved antisense activity. Typically, without being limited by any specified amount, which amounts are offered herein as merely being exemplary of the practice of the invention, administration of approximately 1×10⁵ cells with 0.1 μg of the antisense compound can achieve complete ablation of the target transcript. In addition, the antisense sequence may be less than one fifth the size of the entire length of the transcript. LC-antisense molecule also has high antisense activity with respect to the amount of antisense compound that is administered.

2. LC-antisense compounds can be produced massively with speed, accuracy and cost effectiveness from a bacterial transformant, such as E. coli.

3. The LC-antisense compound-carrier complex is easily absorbed by cells.

4. LC-antisense compounds are stable against nucleases in serum and can form stable complexes with liposomes.

5. LC-antisense compounds are replicated by DNA polymerase in bacterial cells such as E. coli.

6. Ablation of multiple target mRNA is achievable. A chimeric LC-antisense compound may contain a plurality of target-specific antisense sequences in a single vector. The length of each of the antisense sequences may be typically much longer than those of chemically modified antisense oligonucleotides. Several distinct antisense sequences can be located in series. Therefore, it is possible to target multiple types of transcripts of several different genes. This property can be of use in eliminating expression of multiple genes in incurable diseases such as advanced types of cancer exhibiting aberrant gene expression of multiple genes.

7. LC-antisense compounds show low toxicity. Since LC-antisense compounds are composed of the same base composition found in nature, non-specificity and undesired toxic effects are reduced when compared with chemically modified AS-oligos.

8. A random gene or unigene unidirectional antisense library is constructed. Construction of an antisense library with a large number of individual clones may be performed easily and rapidly. A random gene unidirectional antisense library specific to a particular disease can be easily constructed by employing diseased cells or tissues. The random gene antisense library can comprise antisense molecules to disease-specific genes that are not individually verified for their DNA sequences, and thus may be partially redundant. That is, there may be duplicate antisense compounds in the library. On the other hand, the unigene unidirectional antisense library comprises a plurality of sequence-verified genes belonging to any organism at all. The unigene, unidirectional library may be constructed without redundancy among its member antisense compounds. Each member of the unigene library may be cloned separately into an antisense compound-generating vector. These antisense libraries may include thousands or tens of thousands of cloned genes that may be employed for efficiently performing massive gene functionalization by knock-down of gene expression in particular cell types.

Unigene Unidirectional Antisense Library

The present invention provides methods for the construction of a unigene unidirectional antisense library using the phage genome. For constructing the antisense libraries, sequence-verified ‘unigene’ cDNA was used.

The cDNA for the unigene library may be obtained by first cloning the gene using RT-PCR method. In some cases, the cDNAs may be commercially available. The unigene target nucleotide sequence can be selected from genes involved in various malignancies, initiation and progression of immune diseases, infectious diseases, metabolic diseases and hereditary diseases caused by abnormal expression of the gene. For instance, cDNA of TNF-α, NF-kB, c-myc, c-myb, k-ras, raf CD1, CDK2, CDK4, CDK6, cyclin E, TGF-β, c-jun and c-fos are some of these candidate genes.

Without being limited to using any particular phage system, in one embodiment, LC-antisense compounds are produced massively from a bacterial culture containing recombinant bacteriophages. For this purpose, the present inventors cloned cDNA fragments into the multiple-cloning site of the M13 phagemid. Competent bacterial cells were then infected with helper phages to rescue LC-antisense compounds.

A representative procedure for constructing a unigene antisense library is as follows, with the understanding that specific embodiments and exemplifications are presented without limiting the invention in any way thereby:

(1) preparing cDNA fragments of target unigene nucleotide sequence. Thus, a pool of unigenes may be obtained by applying RT-PCR procedure using a pair of specific primers;

(2) cloning the cDNA fragment into a phagemid vector, which is capable of producing LC-antisense compounds. Phagemid vectors containing the F1 replication origin of the filamentous phage were employed for cDNA cloning depending on experimental needs. These include pUC, M13mp, pBlueScript II, pCR2.1, pGEM-f, pGL-2, pβgal, pSPORT and their derivatives;

(3) introducing the recombinant phagemid into competent bacterial cells to make bacterial transformants; and

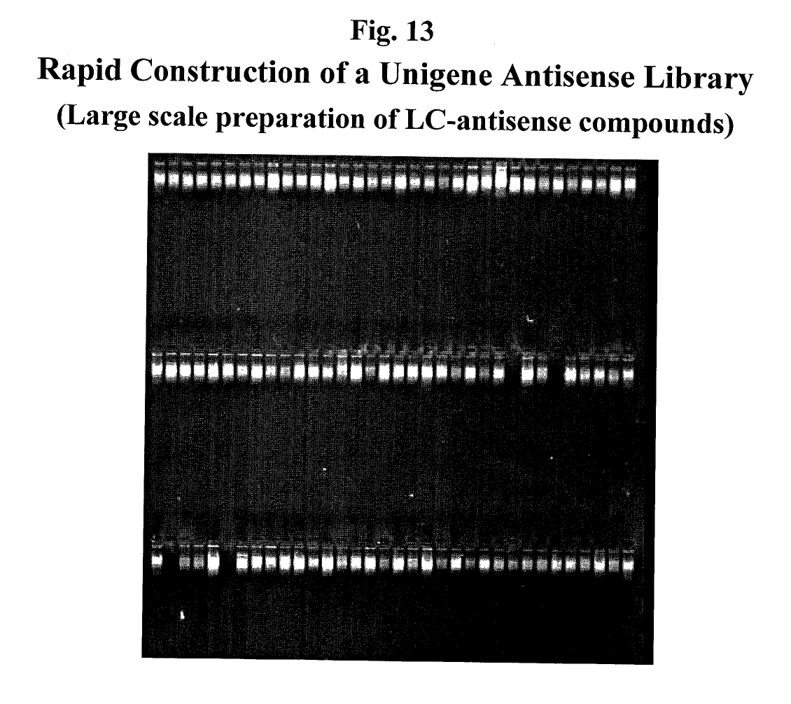

(4) producing an LC-antisense compound library by coinfecting the transformants with helper phage, resulting in mass production of LC-antisense compounds (FIG. 13). All phagemid vectors with the F1 (+) or F1 (−) origin are able to produce LC-antisense compounds.

Massive Functional Genomics

The present invention also provides a high-throughput system for functional genomics using the unigene antisense library discussed above. The functional genomics system of the present invention may be used to rapidly and massively search for gene function. Thus, the antisense library may be used not only for analyzing gene function but it may be used also for target validation as well as for determining the interrelationships among different gene products.

One of the advantages of using the phage genomic library for functional genomics is that it is not necessary to perform a preliminary expression profiling. The unigene antisense library can be directly employed to identify genes critical for a specific disease. This means that a panel of LC-antisense compounds may be used to determine genes that are responsible for the change in a phenotype of a particular cell type by target-specific knock-down of relevant gene expression at least temporarily on a massive and parallel scale. Thus, effective antisense macroarray configurations are possible.

The LC-antisense library may be applied to a single cell type for functional assays. When a defined number of LC-antisense molecules are chosen for transfection, a panel of different types of cells can be used to detect antisense effects for comparative functional profiling.

A representative massive functional genomics protocol may be as follows, with the understanding that specific embodiments and exemplifications are presented without limiting the invention in any way thereby and the steps are not necessarily in the order presented:

(1) preparing a large panel of phage genomic antisense compounds (antisense library), and configuring an antisense macroarray using the antisense library;

(2) aliquoting and plating a type of disease cell or a cell line of interest for each set of multi-well plates;

(3) forming the LC-antisense compound-carrier complexes from the phage genomic antisense library;

(4) transfecting the LC-antisense compound-carrier complexes into the cells in multi-well plates, and carrying out primary functional assays; and

(5) performing additional functional assays for genes that are selected based on the results obtained.

The cells for the transfection of the antisense library may be chosen from cells of interest, including, but not limited to, cells of various types of cancer, such as liver cancer, lung cancer, stomach cancer, breast cancer, colon cancer, pancreatic cancer, ovarian cancer, kidney cancer, bladder cancer, rectal cancer, prostate cancer, skin cancer, as well as cells of obesity, hair follicles of baldness, auto-immune disorders, and metabolic disorders. Cells are seeded in wells in either suspensions or adhesive compositions depending on the cell types and properties being assayed.

The LC-antisense compounds may be complexed with carriers to deliver the antisense compounds into the cells of interest. The ratio of the antisense compounds to carriers may vary based on the types of cells and the types of antisense compounds that are used.

The carriers may be, but not limited to, liposomes, cationic polymers, a complex formed between cationic polymer and viral vectors, HVJ-liposomes, lipofectamine analogs, pronase complexes, peptides, and viral vectors. The antisense compounds may be delivered into cells either alone or complexed with the carrier composition. The LC-antisense compound-carrier complexes are mixed with cells in the multi-well plates, and the LC-antisense compounds in each well are unique in their sequence. Thus, a specific gene of interest is targeted.

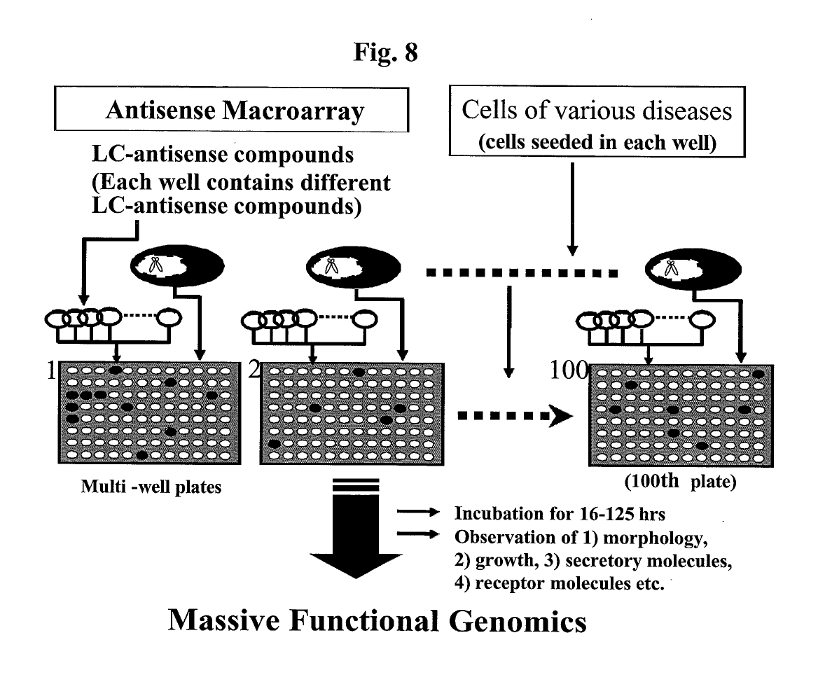

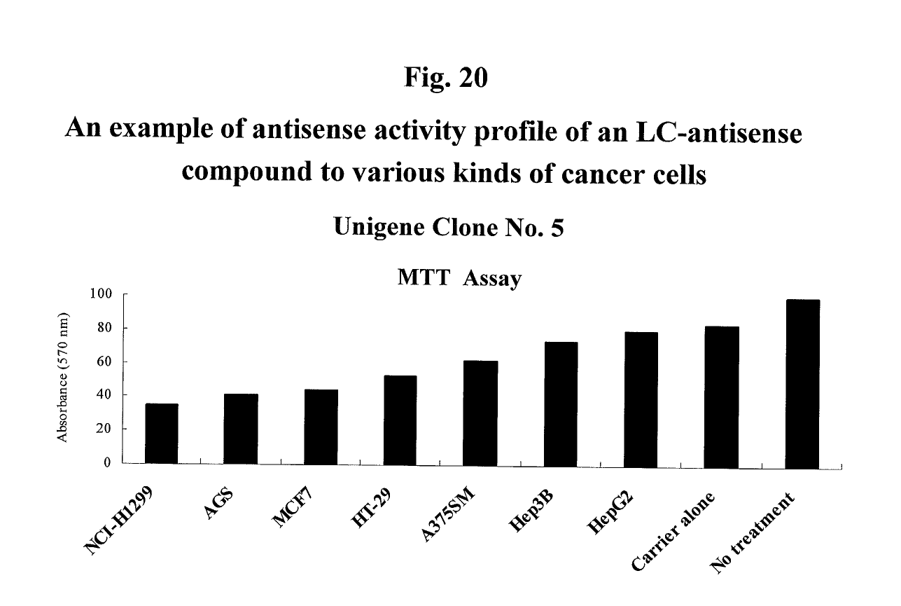

The functional genomics methods described above use a defined set of chosen LC-antisense compounds applied to many types of disease cells or cells of special interest. Thus, the antisense macroarray assembly is intended for functional study in a definitive and comparative manner (FIGS. 8 and 20). The macroarray assembly may be used also for functional diagnostics, to find genes for effective gene therapy, and to examine relationships among genes in the disease cells by comparing their gene functions in the same or similar cells, or from cells of a distinct lineage.

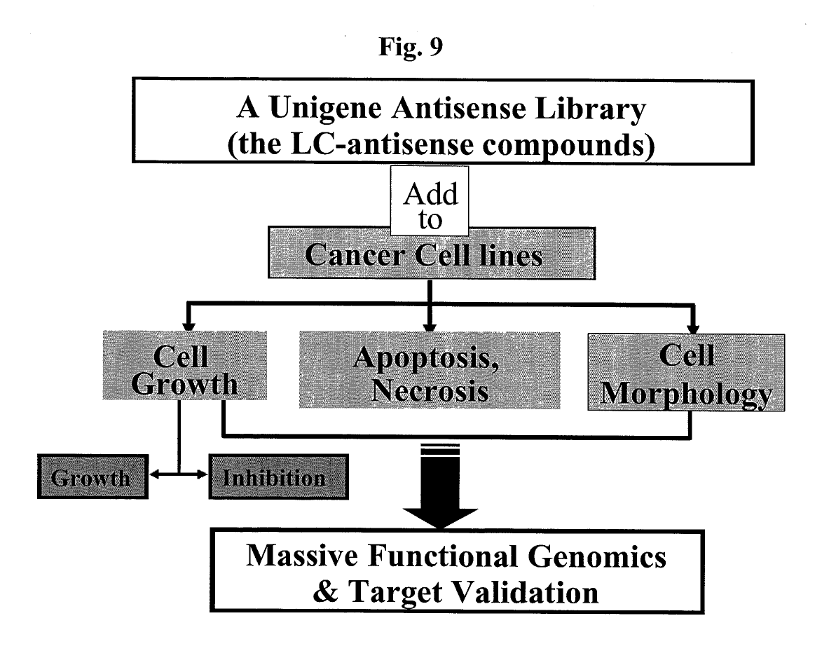

Different gene functionalization assays may be performed. Conventional methods may be employed for gene functionalization assays in carrying out the method of the present invention (FIG. 9). As examples, morphological observation of cells, growth pattern (growth promotion or inhibition) and cell death were used to score parameters of primary assays.

In addition, the present invention provides a system for gene characterization and functionalization on a massive scale using diverse types of cells treated with an antisense macroarray with a limited number of antisense compounds chosen from the phage genomic antisense library.

Cells of interest are seeded in 96- or 384-well plates and incubated for a day in a CO₂ incubator to prepare for treatment with LC-antisense compound-carrier complexes. When the 96-well plates are used in a transfection protocol, the LC-antisense compound to liposome ratio for complex formation can be either 1:3 (w/w), 1:4, or any other adequate ratio depending on the type of cells or liposomes used, and which may be experimentally determined. In general, for the present invention, the ratio of 1:3 (w/w) was employed for efficient transfection.

To study the antisense activity in the transfected cells, the cell culture extract may be conveniently used for immunologic assays. Also, the transfected cells may be used to prepare RNA, which may be used as a template for RT-PCR and Northern blotting. Several other properties such as cell morphology, cell death, growth patterns, and substrate response may be the subject of primary functional studies. Typically, such primary functional studies make use of microscopic observations. See FIG. 9.

Based on the results of primary gene functionalization assays, further assays are carried out to confirm the primary function using techniques in the fields of molecular biology, cell biology, immunology, biochemistry, animal experimentation and the like. These results allow a more precise understanding of the relationships among these genes.

Functional genomics assays may be performed using different assays for specific genes. The following approaches may be preferably employed without limitation:

(1) measuring antisense activities to gene expression by (a) RT-PCR to detect mRNA levels, (b) Western blotting to detect protein levels, and (c) other assays for enzymatic or immunologic reactions;

(2) measuring cell growth and differentiation using MTT assay, thymidine incorporation, and colony formation on soft agarose, including measuring factors associated with DNA replication or chromatin activation (e.g. histone acetylase);

(3) measuring apoptotic cell death, which may be scored for gene function by morphological change, condensation of nucleus, DNA fragmentation, quantitative analysis of apoptosis, intracellular signaling for apoptosis and so on; and

(4) measuring cell cycle regulation, which may be scored by flow cytometry analysis, activities of factors involved in cell cycle progression or pause, and by complex formation between factors involved in cell cycle.

In addition to the above methods, other methods for functional genomics assays using antisense inhibition techniques include assays using molecular biological, biochemical, and physiological changes in vitro and in vivo.

The phage vector allows easy production of the long single-stranded sequence that encompasses the antisense sequence with high sequence fidelity. The new antisense molecules, even with their unconventionally long length, exhibited good sequence specificity in eliminating expression of target mRNA. Without being bound by any particular theory or mechanism of action of the antisense nucleic acid, it is thought that once a small portion of the antisense sequence binds to its complementary sequence, the antisense sequence zips through the entire length of the complementary target sequence. The lengthy duplex formed between the antisense DNA and sense RNA is then much more stably maintained as a substrate for RNaseH activity.

Another reason for the advantageous binding of the inventive antisense molecule may be that there may exist a higher chance for the long antisense molecule to bind to a target site that is structurally exposed. Messenger RNA tends to form extensive secondary and tertiary structures within its own sequence and by interaction with RNA binding proteins in the cell cytoplasm. Finding an open target site for an antisense molecule is critical for successful antisense activity. With its long length, the phage genomic antisense molecule has to have some sequence that can access exposed complementary sequences of target mRNA, thus improving the chances for target mRNA ablation.

Antisense Molecular Therapy

The inventive LC-antisense molecules are effective therapeutic agents against various types of cancer, viral infection, immunologic disorders, metabolic disorders and other human diseases in which modulation of gene expression can be beneficial to intervene in disease initiation and progression.

The principles of the antisense molecules of the invention may be applied to any target gene of interest. While TNF-α and NF-κB specific LC-antisense compounds are disclosed as examples of the antisense molecule of the invention, the antisense molecule of the invention may be made against any gene of interest. In fact, the LC-antisense molecules of the invention were significantly more stable to nucleases and were effective in target ablation. Exemplified sequence specific reduction of the TNF-α and NF-κB target genes supports the broad utility of an antisense molecular therapy method. Thus, the antisense molecule of the invention may be used to bind to any endogenously expressed target transcript from any source.

Antisense activity was also examined at the protein level to ensure correlation of both target mRNA and protein elimination. Administration of TNFα-M13AS was found to significantly reduce rat TNF-α secretion in cell culture media, confirming effective antisense activity. In contrast, control phage genomic compounds (single-stranded circular molecules without an antisense insert) exhibited only a mild reduction in TNF-α secretion. The slight decrease of TNF-α secretion by the addition of control antisense molecule can be explained, in part, by the cytotoxicity of free cationic liposomes deposited inside endosomes. Cells treated with cationic liposomes alone exhibited lower viability than cells with liposome-antisense molecule complex.

In therapeutic applications, the large circular nucleic acid molecules can be formulated for a variety of modes of administration, including oral, topical or localized administration. Techniques and formulations generally may be found in Remington's Pharmaceutical Sciences, Mack Publishing Co., Easton, Pa., latest edition. The active ingredient that is the antisense molecule is generally combined with a carrier such as a diluent of excipient which may include fillers, extenders, binders, wetting agents, disintegrants, surface-active agents, erodable polymers, or lubricants, depending on the nature and mode of administration and dosage forms. Typical dosage forms include tablets, powders, liquid preparations including suspensions, emulsions and solutions, granules, and capsules.

Certain of the large circular nucleic acid compounds of the present invention may be particularly suited for oral administration which may require exposure of the drug to acidic conditions in the stomach for up to about 4 hours under conventional drug delivery conditions and for up to about 12 hours when delivered in a sustained release form. For treatment of certain conditions it may be advantageous to formulate these antisense compounds in a sustained release form.

Systemic administration of the large circular nucleic acid molecules may be achieved by transmucosal or transdermal means, or the compounds can be administered orally. For transmucosal or transdermal administration, penetrants appropriate to the barrier to be permeated are used in the formulation. Such penetrants are generally known in the art, and include, for example, bile salts and fusidic acid derivatives for transmucosal administration. In addition, detergents may be used to facilitate permeation. Transmucosal administration may be through use of nasal sprays, for example, as well as formulations suitable for administration by inhalation, or suppositories.

The large circular nucleic acid molecule of the present invention can also be combined with a pharmaceutically acceptable carrier for administration to a subject. Examples of suitable pharmaceutical carriers are a variety of cationic lipids, including, but not limited to N-(1-2,3-dioleyloxy)propyl)-n,n,n-trimethylammonium chloride (DOTMA) and dioleoylphophotidyl ethanolamine (DOPE). Liposomes are also suitable carriers for the antisense molecules of the invention. Another suitable carrier is a slow-release gel or polymer comprising the claimed antisense molecules.

The large circular nucleic acid molecules may be administered to patients by any effective route, including intravenous, intramuscular, intrathecal, intranasal, intraperitoneal, intratumoral, subcutaneous injection, in situ injection and oral administration. Oral administration may require enteric coatings to protect the claimed antisense molecules and analogs thereof from degradation along the gastrointestinal tract. The large circular nucleic acid molecules may be mixed with an amount of a physiologically acceptable carrier or diluent, such as a saline solution or other suitable liquid. The antisense molecules may also be combined with other carrier means to protect the nucleic acid molecules or analogs thereof from degradation until they reach their targets and/or facilitate movement of the antisense molecules or analogs thereof across tissue barriers.

In one embodiment, the large circular nucleic acid molecules are administered in amounts effective to inhibit cancer or neoplastic cell growth. In other embodiments, the antisense molecule may be used to treat viral infections, such as, but not limited to herpes, human papilloma virus (HPV), HIV, small pox, mononucleosis (Epstein-Barr virus), hepatitis, respiratory syncytial virus (RSV) and so on. In addition, metabolic diseases, such as, but not limited to, phenylketonuria (PKU), primary hypothyroidism, galactosemia, abnormal hemoglobins, types I and II diabetes, obesity and so on are also targets. The inventive antisense molecule may be used to treat other diseases such as immunologic diseases including such diseases as, but not limited to, Sjogren's Syndrome, antiphospholipid syndrome, immune complex diseases, Purpura, Schoenlein-Henoch, immunologic deficiency syndromes, systemic lupus erythematosus, immunodeficiency, rheumatism, and so on.

The actual amount of any particular large circular nucleic acid molecule administered will depend on factors such as the type and stage of the disease or infection, the toxicity of the antisense molecule to other cells of the body, its rate of uptake by the cells, and the weight and age of the individual to whom the nucleic acid molecule is administered. An effective dosage for the patient can be ascertained by conventional methods such as incrementally increasing the dosage of the antisense molecule from an amount ineffective to inhibit cell proliferation to an effective amount. It is expected that concentrations presented to the diseased cells may range from about 0.1 nM to about 30 μM will be effective to inhibit gene expression and show an assayable phenotype. Methods for determining pharmaceutical/pharmacokinetic parameters in chemotherapeutic applications of antisense molecules for treatment of cancer or other indications are known in the art.

The large circular nucleic acid molecules are administered to the patient for at least a time sufficient to have a desired effect. To maintain an effective level, it may be necessary to administer the antisense nucleic acid molecules several times a day, daily or at less frequent intervals. For cancer cells, antisense molecules are administered until cancer cells can no longer be detected, or have been reduced in number such that further treatment provides no significant reduction in number, or the cells have been reduced to a number manageable by surgery or other treatments. The length of time that the antisense molecules are administered will depend on factors such as the rate of uptake of the particular molecule by cancer cells and time needed for the cells to respond to the molecule.

The following examples are offered by way of illustration of the present invention, and not by way of limitation.

### EXAMPLES

### Example 1

### Contruction of LC-Antisense Compounds Using M13 Bacteriophage

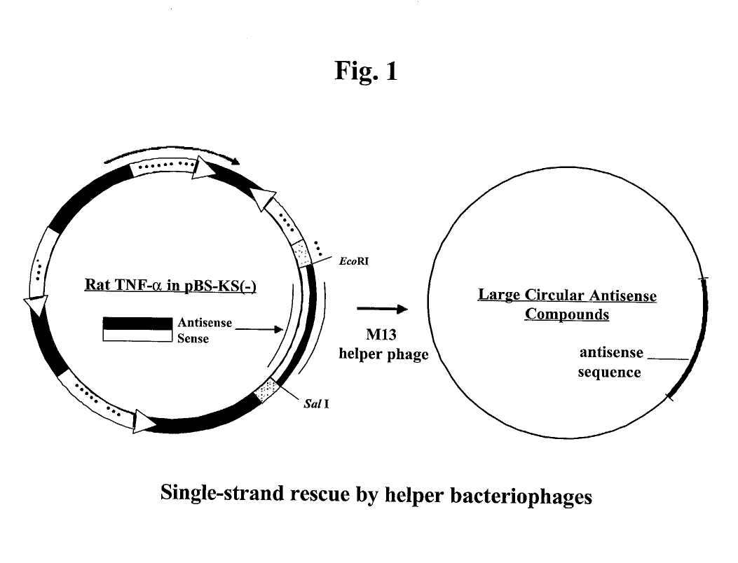

Experiments were carried out to determine whether the circular phage genome of M13 bacteriophages (phage) can harbor an antisense sequence as a part of its genome and whether these new antisense molecules can overcome the problems associated with synthesized forms of antisense oligonucleotides. Production of recombinant M13 phage was carried out by infecting M13K07 helper phages into bacterial cells that were already transformed with pBluescript KS (−) phagemid (Jupin et al. Nucleic Acid Res., 23, 535-536 (1995)). We utilized the F1 origin of the phagemid to generate single stranded circular phage genome containing either antisense or sense sequence for a target gene. In the case of the gene encoding rat TNFα, the entire cDNA of the gene was placed into pbluescript KS (−) vector to produce the antisense sequence (FIG. 1).

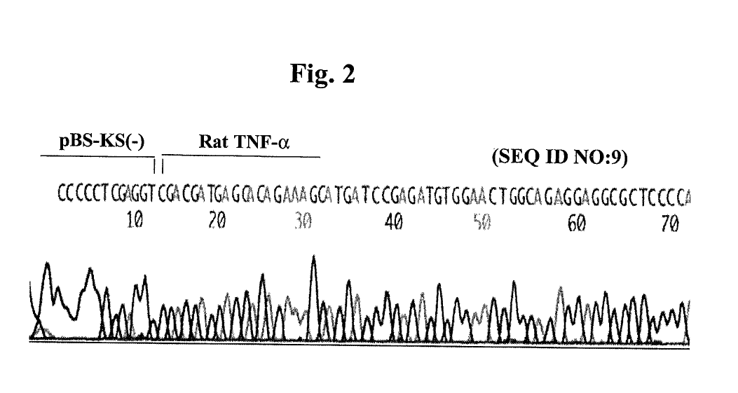

The antisense sequence in the single-stranded genomic DNA was confirmed by DNA sequencing using T7 sequencing primers (FIG. 2). Both the 5′ and 3′ flanking sequences of the TNF-α antisense insert were shown to be those of the phagemid vector. The insert sequence corresponded with that of TNF-α mRNA, demonstrating that the antisense sequence was present. The circular phage genome containing the antisense sequence for TNF-α and NF-kB were designated as TNFα-M13AS and NFκB-M13AS, respectively.

1. mRNA Induction and Cloning of Genes Encoding Rat TNF-α and Human NF-κB

Rat TNF-α expression was induced with lipopolysaccharide (LPS, 30 μg/ml) in WRT7/P2 cells. Cells at 1×10⁶ cells/well were seeded in each well of a 48-well plate and were treated with LPS for 4 to 24 hours. Cells were harvested at desired time points to examine the amounts of mRNA. The LPS incubation time by which TNF-α expression was induced at the highest level was chosen for further experiments. The highest level of rat TNF-α expression was determined 6 hours after LPS treatment.

Rat TNF-α cDNA was obtained from the amplified cDNA fragments as described above. The RT-PCR fragment (708 bp) of TNF-α that comprises the entire coding sequence was amplified with a pair of PCR primers: 5′-GATCGTCGACGATGAGCACAGAAAGCATGATCC-3′ (SEQ ID NO:1), and 5-GATCGAATTCGTCACAGAGCAATGACTCCAAAG-3′ (SEQ ID NO:2). The rat TNF-α cDNA fragment was cloned into the multiple cloning site of pBluescript (pBS) KS (−) vector using Sal I and EcoR I restriction sites in the same direction as the lacZ gene (FIG. 1).

Similarly, cDNA fragments of the NF-kB gene was amplified with a pair of PCR primers and cloned into the EcoRV site of pBS-KS (+) vector after blunting the ends. Amplified cDNA fragments were always confirmed with both restriction digestion and DNA sequencing.

In detail, THP1 cells derived from leukocytic monocytes which were transfected with NFκB-M13AS, NFκB-M13SE or M13SS complexed with liposomes in a ratio of DNA to liposome ratio of about 1:4 (w/w) and cultured. One day after lipofection, cells were stimulated with PMA (160 nM) for 6 hours. Total RNA was isolated and subjected to RT-PCR using a pair of primers: 5′-GATCGTCGACGCGCCACCCGGCTTCAGAATGGC-3′ (SEQ ID NO:3) and 5′-GATCGAATTCGGTGAAGCTGCCAGTGCTATCCG-3′ (SEQ ID NO:4). The PCR product was used in Southern blot analysis using a 25mer oligonucleotide probe of 5′-CTTCCAGTGCCCCCTCCTCCACCGC-3′ (SEQ ID NO:5).

2. Construction of Large Circular Nucleic Acid Molecules Employing a Phagemid Vector and the M13K07 Helper Bacteriophages

(1) Construction of single-stranded bacteriophage genome harboring either sense or antisense sequences

Large circular nucleic acid molecules that contain an antisense region specific to the target genes were constructed according to standard cloning procedure (Sambrook et al., Molecular Cloning, 1989). Competent bacterial cells (XL-1 Blue MRF′) containing the pBS-KS (+) or (−) phagemid with the appropriate cDNA were infected with helper bacteriophage M13K07 (NEB Nucleic Acids, USA). The orientation of the cloned cDNA in the phagemid vector determines which of the sense or antisense sequence will be produced. 20% polyethylene glycol (PEG 8000) was added to the supernatant of an overnight culture of helper phage infected cells grown in 2× YT. The bacteriophage precipitate was resuspended in TE (pH 8.0), and phage genomic DNA was isolated by phenol extraction and ethanol precipitation.

(2) Purification ofthe Phage Genomic Antisense Molecules

Purification of phage genomic antisense molecules from the residual genomic DNA of helper bacteriophage and host bacterial cells was carried out either with 0.8% low melting point (LMP) agarose gel for small scale purification or with gel filtration column chromatography (1.0×50 cm) for large scale purification. The column resin for gel filtration was superfine Sephacryl™ S-1000 (molecular cutoff: 20,000 bp) (Amersham Pharmacia Biotech AB, Sweden), and was packaged and equilibrated with 50 mM Tris-HCl buffer containing 0.2 M NaCl (pH 8.3). The starting volume of the antisense molecules was adjusted to 5% of the gel void volume and DNA elution was carried out with the same buffer used for resin equilibration (flow rate: 0.3 ml/min). Samples were UV scanned at 260/280 nm with a dual UV detection system and were collected every 5 min during elution. Sample fractions were washed and precipitated with 70% cold ethanol and were resuspended in distilled ultrapure water and PBS (phosphate buffered saline) for subsequent experiments. The purified antisense molecules were tested for quantity and purity on a 1% agarose gel. Control sense molecules were constructed with the TNF-α cDNA fragment cloned in pBS-KS (+), in the opposite orientation of the lacZ gene in the vector. Single stranded molecules of either sense or antisense were confirmed for sequence integrity by employing the T7 primer for sequencing. DNA sequencing was carried out with an automated DNA sequencer (FIG. 2).

### Example 2

### Structural Analysis and Stability Test of the Phage Genomic Circular Antisense Molecules

1. Single-Stranded Circular TNF-α Antisense Molecules

The fact that the antisense molecules are single-stranded, circular and stable was tested in the following manner. 1 μg LC-antisense molecules containing antisense region targeted to the gene encoding TNF-α were treated with Xho I (10 U/μg DNA), Exonuclease III (160 U/μg DNA), or S1 nuclease (10 U/μg DNA) at 37° C. for 3 hrs, and subjected to phenol extraction, ethanol precipitation and gel electrophoresis on a 1% agarose gel to study their stability as well as digestion patterns.

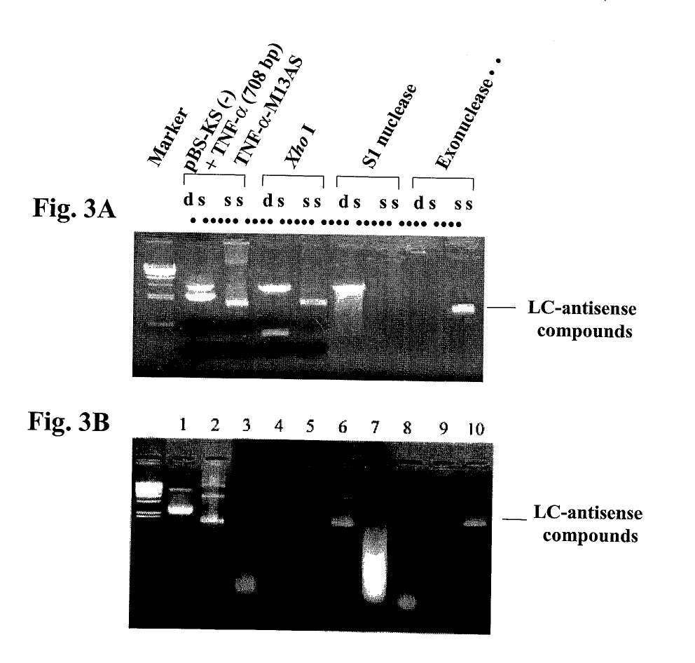

TNFα-M13AS was tested for its circular structure and stability to nucleases. The LC-antisense molecules were expected to be stable to exonucleases because of their closed circular structure. When TNFα-M13AS was incubated with the endonuclease Xho I and exonuclease III, the antisense molecules were found to be largely intact even after a 3 hour incubation with these nucleases (FIG. 3A). In contrast, when Xho I was added to the double stranded replication form of the recombinant M13 phage DNA, the DNA was, as expected, completely digested by the combination of the restriction enzyme and exonuclease III. The single-stranded TNFα-M13AS was also completely digested by S1 nuclease, a nuclease that is specific for single-stranded DNA. Thus, it was confirmed that TNFα-M13AS was shaped as a single-stranded circular molecule.

2. Stabilit Testfor TNFα-M13A

For the stability test, 1 μg of antisense molecules was added alone or after complex formation with liposomes in a ratio of DNA:liposome of about 1:3 (w/w). A not heat inactivated 30% FBS solution was added to the antisense-liposome complex and incubated at 37° C. for varying time periods for up to 48 hours. After incubation with FBS and the nucleases, antisense DNA was extracted with chloroform, precipitated with ethanol and electrophoresed on a 1% agarose gel.

Phage genomic antisense molecules were also found to be stable since their structural integrity was largely preserved after incubation with serum. When TNFα-M13AS was combined with cationic liposomes, a large fraction of the antisense molecules remained intact after extended incubation in fetal bovine serum (FBS). In fact, TNFα-M13AS remained intact even after a 24 hour incubation with 30% FBS (FIG. 3B). The results suggest that the phage genomic antisense molecules may be further stabilized during in vivo application by forming complexes with liposomes.

### Example 3

### Effective and Specific Elimination of Rat TNF-Alpha Expression by TNF_{ALPHA}-M13AS

The antisense activity of TNFα-M13AS was tested. TNFα-M13AS contains a long antisense sequence that includes nonspecific antisense phagemid vector sequences and an antisense region specific to rat TNFα mRNA. The fact that the phage genomic antisense molecules have a large amount of nonspecific sequences necessitates a thorough analysis of target specificity of the antisense activity. In order to determine whether phage genomic antisense molecules act specifically to eliminate target gene expression, multiple control genes were used to compare levels of mRNA ablation.

1. Cell Cultures

Monocytic mouse cell line WRT7/P2 and human cell line THP-1 were maintained in either RPMI 1640 or EMEM (JBI, Korea) supplemented with 10% heat-inactivated FBS (JBI, Korea), 100 μg/ml penicillin and 100 μg/ml streptomycin. Cells were cultured in a CO₂ (5%) incubator at 37° C. and carefully maintained to avoid overgrowth. Cell media was exchanged with fresh culture media the day before lipofection (16 hours) and tested for cell viability with 0.4% trypan blue staining on the day of experiments.

2. Transfection of TNFα-M13AS Complexed with Liposomes

Cationic liposomes, such as Lipofectamine™, Lipofectamine 2000™ or Lipofectamine Plus™ (Life Technologies, USA) were mixed with either antisense molecules or sense control molecules. These liposome-DNA complexes were mixed with OPTI-MEM (Life Technologies, USA), and were then added to cells according to the protocol suggested by the manufacturer.

Lipofection details are as follows. Cells were cultured in RPMI 1640 or EMEM supplemented with 10% FBS and were washed twice with OPTI-MEM 30 minutes prior to lipofection. Cells were seeded in a 48-well plate (1×10⁵ cells/well) in 200 μl of culture media. Antisense molecules were mixed with cationic liposomes in a ratio of about 1:3 (w/w) and added to cells for transfection. Cells were incubated for 6 hours at 37° C. in serum-free media. Following the lipofection, 2× FBS and antibiotics were added to the culture medium and incubated further for 18 hrs at 37° C. Rat TNF-α expression was induced with LPS (30 μg/ml). Cells were used for the preparation of RNA, and culture supernatant was tested for the presence of IL-10 with Enzyme Linked Immuno-Sorbent Assay (ELISA).

3. Detection of Transcription with RT-PCR

RNA preparation was carried out with Tri reagent™ (MRC, USA) according to the protocol recommended by the manufacturer. Cells harvested from each well were mixed with 1 ml Tri Reagent and 200 μl chloroform for RNA purification. Purified RNA was subjected to RT-PCR in a 50 μl reaction volume by using the Access™ RT-PCR kit (Promega, USA). In a PCR tube were added purified RNA, a pair of primers: 5′-CATCTCCCTCCGGAAAGGACAC-3′ (SEQ ID NO:6) and 5′-CGGATGAACACGCCAGTCGC-3′ (SEQ ID NO:7), AMV reverse transcriptase (5 U/μl), Tfl DNA polymerase (5 U/μl), dNTP (10 mM, 1 μl) and MgSO₄ (25 mM, 2.5 μl). Reverse transcription and polymerase chain reaction were sequentially carried out in a thermal cycler (Hybaid, UK). Synthesis of the first strand cDNA was carried out at 48° C. for 45 min and subsequent DNA amplification was carried out in 30 repetitive cycles, at 94° C. for 30 sec (denaturation), 59° C. for 1 min (annealing), and 68° C. for 2 min (polymerization). PCR product was confirmed on a 1% agarose gel, and quantitative analysis of the amplified DNA was performed with AlphaImager 1220, a gel documentation apparatus (Alpha Inno-Tech corporation, USA).

4. Southern Blotting

Probes for Southern hybridization were prepared with ECL (enhanced chemical luminescence) oligo-labeling and detection system (Amersham Life Science, UK). RT-PCR products were run on a 1% agarose gel and transferred onto a nylon membrane in 0.4 M NaOH solution. An oligonucleotide probe for TNF-α was a 22 mer: 5′-GATGAGAGGGAGCCCATTTGGG-3′ (SEQ ID NO:8), and an oligonucleotide probe for NF-κB was a 25 mer: 5′-CTTCCAGTGCCCCCTCCTCCACCGC-3′ (SEQ ID NO:5).

Oligonucleotide probes of 100 pmol were mixed with fluorescein- 11-dUTP, cacodylate buffer and terminal transferases, and were incubated at 37° C. for 70 min for ECL labeling. Probe hybridization to a nylon membrane with transferred DNA was carried out in a 6 ml hybridization buffer (5× SSC, 0.02% SDS, liquid block) at 42° C. for 14 hrs. The nylon membrane was washed twice in 5× SSC containing 0.1% SDS and once in 1× SSC containing 0.1% SDS, at 45° C. for 15 min for each washing. The membrane was incubated with an antibody conjugated to HRP anti-fluorescein for 30 min, followed by incubation with ECL detection reagent for about 5 min before exposure to an X-ray film.

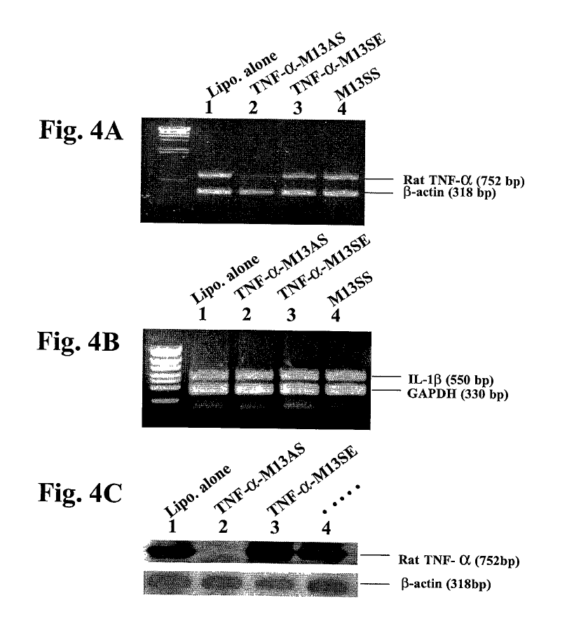

To test the specific activity of TNFα-M13AS, 0.5 μg (1.4 nM) of the antisense molecules were complexed with 1.5 μg of cationic liposome and were added to 1×10⁵ cells of a monocytic cell line, WRT7/P2. The cells were then induced for TNF-α expression by LPS treatment. When the cells were treated with TNFα-M13AS, the induction level of TNF-α mRNA was significantly reduced. In contrast, when cells were treated with either TNFα-M13SE (the sense strand of TNF-α) or M13SS (single-stranded phage genome without the antisense insert) they did not show much reduction of TNF-α mRNA (FIGS. 4A and 4C). RT-PCR band of TNF-α was confirmed by Southern hybridization using a probe that binds to the middle of the amplified DNA fragments.

TNFα-M13AS contains the rat TNF-α antisense sequence as well as antisense sequences of the β-galactosidase (LacZ) and the β-lactamase (Amp) genes, harboring a total of 3.7 kb single stranded circular genome. The TNF-α specific antisense portion is about 708 bases long. Thus, the TNF-α specific antisense sequence in TNFα-M13AS is itself very long when compared with conventional synthetic antisense molecules of some 20 or 30 nucleotides. This is significant because it has been generally believed in the art that as the antisense molecule is lengthened, its sequence specificity declines. Further confirming tests were carried out to show that the antisense activity of TNFα-M13AS is indeed sequence specific.

In order to demonstrate sequence specific antisense activity, three different genes were examined for mRNA levels after lipofection of TNFα-M13AS. These were β-actin, GAPDH (glyceraldehyde 3-phosphate dehydrogenase), and IL-1β (interleukin-1 β). Expression of these genes was not affected by lipofection of TNFα-M13AS (FIGS. 4A-4C).

Dose response of TNFα-M13AS in its antisense activity was also examined. When TNFα-M13AS was used at a concentration of 0.01 μg (0.03 nM), TNF-α expression was only slightly reduced. At a concentration of 0.05 μg (0.14 nM), TNF-α expression was partially eliminated. When the amount of TNFα-M13AS was increased to 0.1 μg (0.28 nM), TNF-α mRNA was found to be completely abolished. These results show that TNFα-M13AS is effective for the elimination of target mRNA using a much smaller amount than conventionally used antisense molecules.

### Example 4

### Expression Patterns of Rat TNF_{ALPHA} Protein

Quantitation of target proteins after antisense treatment was examined with either ELISA or Western blotting method. For the ELISA assay, cell culture supernatant was diluted 50 fold and added to an ELISA plate coated with antibody against TNF-α. Biotinylated secondary antibody to anti TNF-α was added into each well of the ELISA plate and incubated at room temperature for 90 minutes. After three washings, streptavidin-peroxidase was added, and incubated for 45 minutes. The plate was washed four times to remove unbound streptavidin-peroxidase, and chromogen was added. After a 20 minute incubation for color development, optical density was measured at 450 nm.

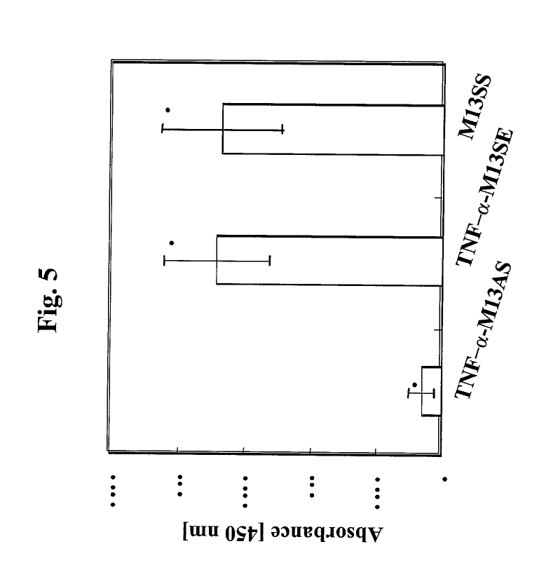

WRT7/P2 cells were lipofected with TNFα-M13AS, and TNF-α secreted from the transfectants was measured using the ELISA assay. Similar to the level of reduction of endogenous TNF-α mRNA, TNF-α protein in the cell culture supernatant was also reduced by more than 90% after administering TNFα-M13AS (FIG. 5). However, neither of the control antisense molecules, TNFα-M13SE (containing the sense strand of the TNF-α gene) or M13SS, reduced TNF-α expression in WRT7/P2 transfectants. These results demonstrate that TNFα-M13AS was effective in both the elimination of TNF-α mRNA and subsequent disappearance of TNF-α from the transfectants.

### Example 5

### Effect of NF_{KAPPA}B-M13AS on Human NF_{KAPPA}B Transcription

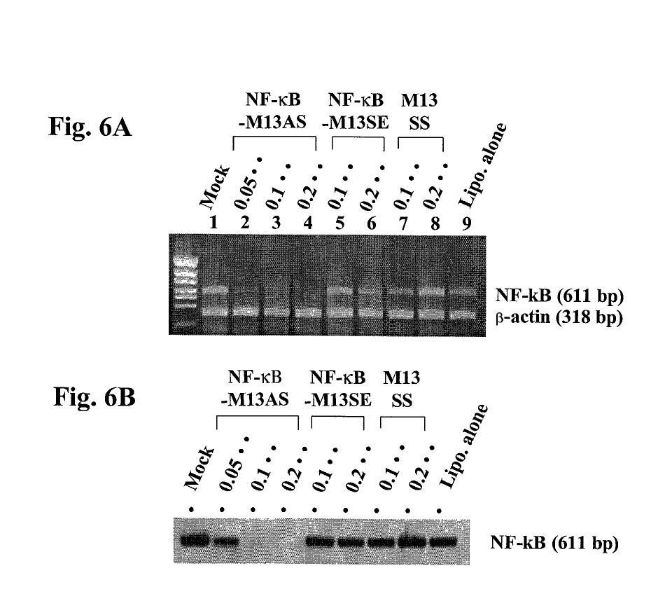

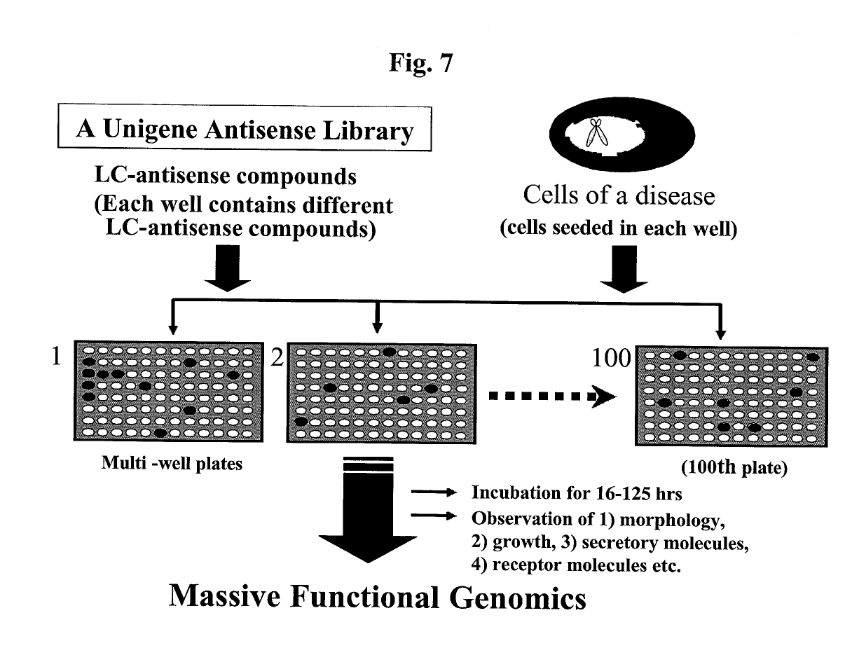

Observing the effectiveness of TNFα-M13AS, experiments were carried out to determine whether phage genomic antisense compounds specifically directed to other genes block the expression of another gene, such as NF-κB. Antisense compound to NF-κB (NFκB-M13AS) was produced and tested in THP-1 cells for efficient antisense activity. NFκB-M13AS was also complexed with liposomes and was added to the cells in increasing amounts. When 0.05 μg (0.14 nM) of NFκB-M13AS was added to THP-1, NF-κB mRNA was reduced by about 70%. When the amount of NFκB-M13AS was increased to 0.1 μg (0.28 nM) and to 0.2 μg (0.56 nM), NF-κB mRNA was eliminated by more than 90%. In contrast, cells that were treated with either NFκB-M13SE (phage genomic DNA with the sense sequence of NFκKB) or with M13SS, NF-κB expression was not much affected (FIGS. 6A-6B).

### Example 6

### Construction of a Unigene Antisense Library

1. Construction of a Unigene cDNA Library

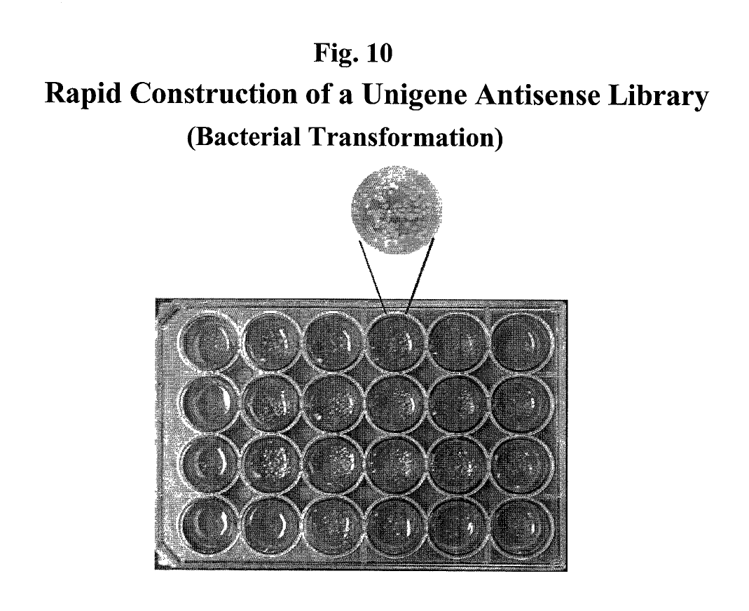

To construct a unigene antisense library as an example, 687 unigene cDNA fragments among tens of thousands of unigene clones were subcloned into the multiple cloning site of pBluescript (pBS) SK(−) vector in the same direction as the LacZ gene. Epicurian Coli® XL-10 Gold Ultracompetent cells (Stratagene, USA) were transformed with the recombinant phagemid by the calcium-chloride method (FIG. 10).

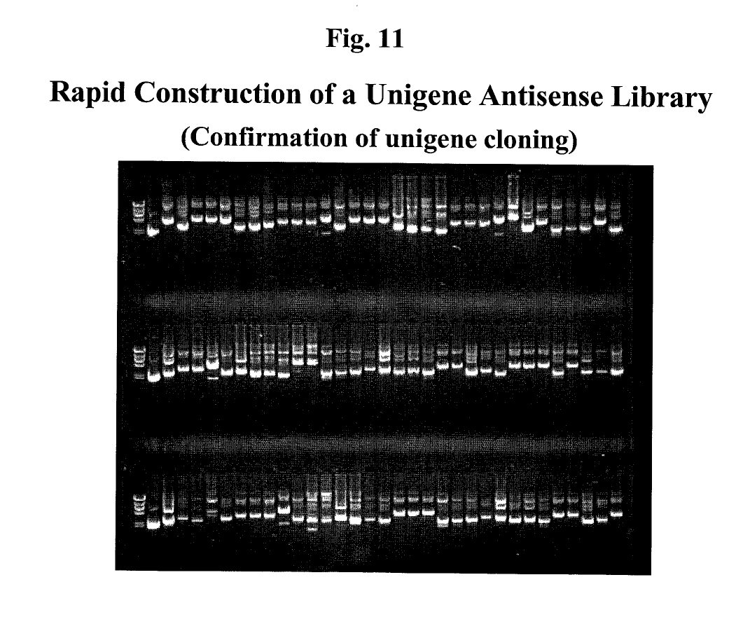

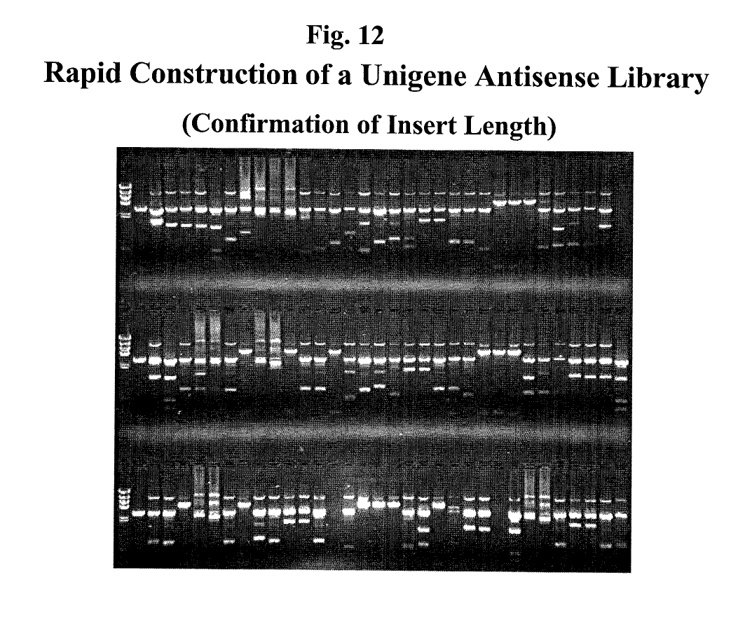

The recombinant phagemids were purified from the transformants by akaline-SDS method in a high-throughput manner (FIG. 11), and were digested with the same restriction enzymes used in the subcloning process, followed by agarose gel electrophoresis (FIG. 12).

2. Making Unigene LC-Antisense Compounds

Bacterial culturing and purification steps for making unigene antisense library were performed as follows. Competent bacterial cells containing pBS SK(−) phagemid with a cDNA insert were plated on LB agar plates containing 50 μg/ml of ampicillin and 50 μg/ml of tetracycline and incubated at 37° C. for 16 hours. Isolated single colonies that were seeded in each well of 96- deep well plate, were aliquotted with 1.5 ml 2× YT liquid media (tryptone 16 g, yeast extract 10 g, NaCl 10 g per 1000 ml) containing 50 μg/ml ampicillin, and precultured for 7 hrs at 37° C. with vigorous shaking. To produce LC-antisense compounds from each phagemid, 20 μl of the preculture was multi-channel pipetted to the wells prefilled with 1.4 ml 2× YT liquid media free of ampicillin, but which also contained 9 μl of helper bacteriophage M13K07 (NEB Nucleic Acids, USA).

After a 1 hour incubation, 4.2 μl of 50 μg/ml kanamycin was added and cultured for 12 hours under the same conditions described above. The infection was carried out in triplicate for each clone to maximize the yield of antisense molecules in a single purification step.

For high-throughput massive production of single-stranded LC-antisense molecules, 20% polyethylene glycol (PEG 8000) was added to culture supernatant of the overnight culture using QIAprep 96 M13 Kits (Qiagen, German). Purification steps were performed with a QIAVAC Vacuum Manifold (Qiagen, German) following manufacturer's instructions.

Purified antisense molecules were run together with control molecules derived from pBS SK(−) phagemid without a cDNA insert on a 1% agarose gel to test the quantity and purity of the antisense molecules (FIG. 13).

After confirming the integrity of purified antisense compounds by gel electrophoresis, the unigene antisense library of 687 species of LC-antisense compounds was placed in eight 96-well plates.

### Example 7

### Lipofection of a Unigene Antisense Library Into Lung and Liver Cancer Cells

This is an example of applying the antisense library to determine genes that are involved in the disease process of a particular cell line. By the principle that specific binding of antisense library molecules to the complementary mRNA sequence can inhibit the expression of the target gene, the present inventors first screened antisense compounds affecting the growth of liver and lung cancer cells by lipofection unigene antisense library into liver and lung cancer cell lines.

Cancer cell lines, HepG2 and NCI-H1299, were obtained from Korean cell line bank (KCLB, Korea). The cell lines were maintained in DMEM media (JBI, Korea) supplemented with 10% heat-inactivated FBS (JBI, Korea), 100 μg/ml of penicillin and 100 μg/ml of streptomycin.

After washing the cells twice with OPTI-MEM (Life Technologies, USA), 7×10³ cells of HepG2 and 5×10³ cells of NCI-H1299 were seeded in each well of the thirteen 96-well plates in 100 μl of optimal culture media supplemented with 10% FBS. The cells were incubated for 12-18 hours at 37° C. in a 5% CO₂ incubator. 0.1 μg of each LC-antisense compound that was to be transferred into the eight 96-well plates was complexed with 0.3 μg of cationic liposomes, and the LC-antisense compound-carrier complex was added to the cultured cells. Cell media were changed with fresh media 24 hours after transfection and incubated for 3 to 4 days.

To compare the effects of the LC-antisense compounds on cell proliferation, identical quantities of carrier alone and control DNA-carrier complexes were also added to the cells in a different 96-well plate and assayed simultaneously. Control DNA was a large circular phage genomic DNA without a cDNA insert. HepG2 and NCI-H1299 cells were then incubated for 3 and 4 days, respectively.

### Example 8

### Screening for Genes Critical for Growth of Liver and Lung Cancer Cells

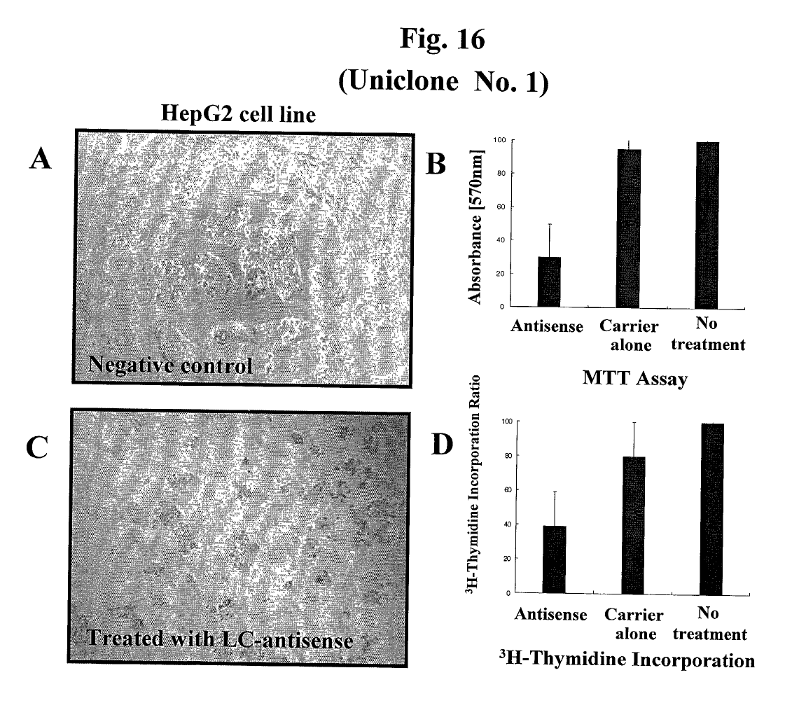

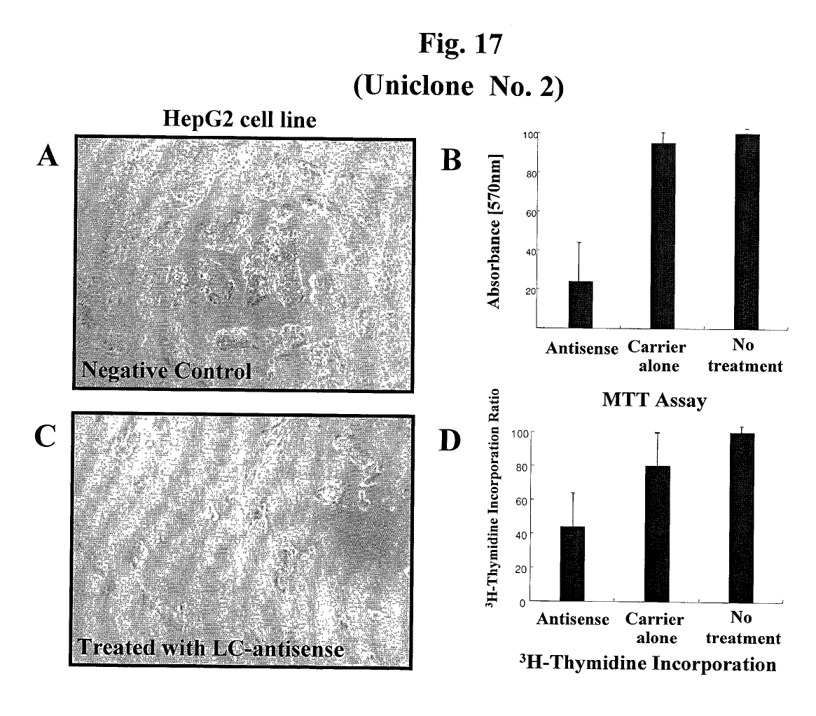

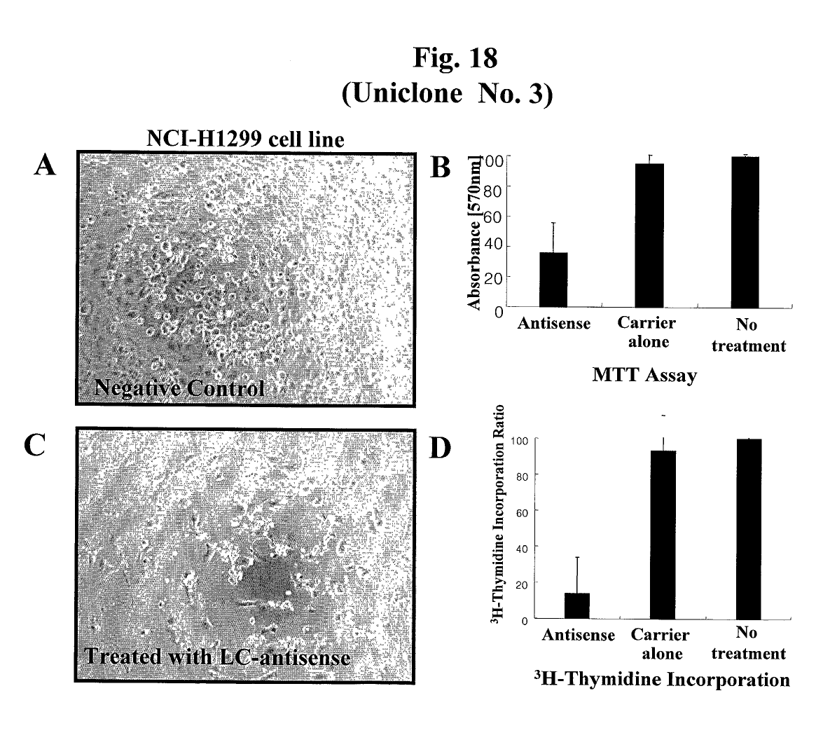

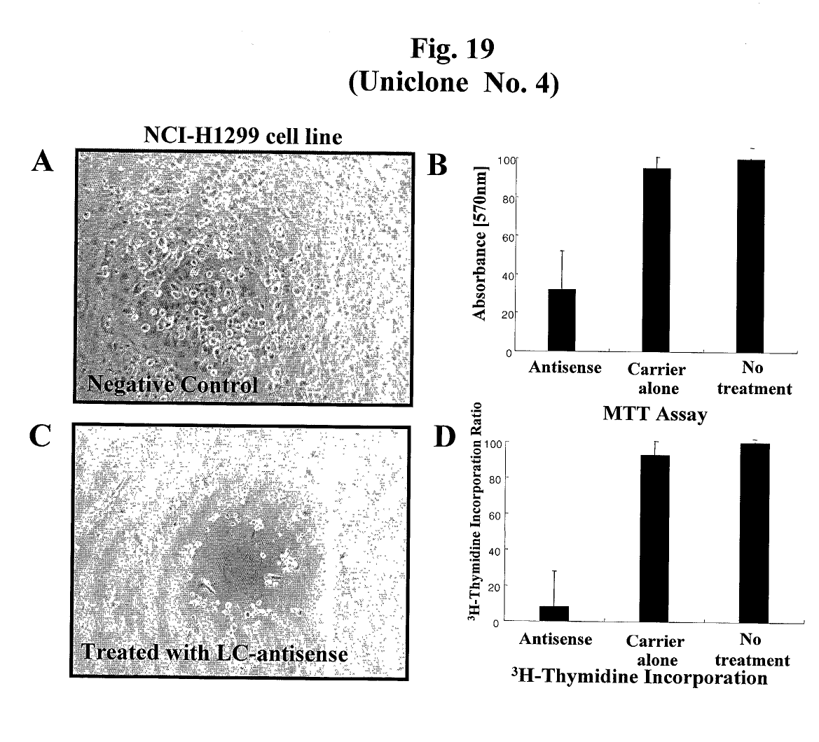

In order to screen for genes involved in the growth of liver and lung cancer cells, light microscopy, MTT reduction assay and [³H]-thymidine incorporation assays were performed. Growth inhibition of liver and lung cancer cells using LC-antisense compounds was first confirmed by light microscopy (original magnification, ×200) (FIGS. 16-19).

For the MTT reduction assay, at 3 to 4 days after the transfection of LC-antisense compounds, cell culture media was replaced with 50 μl of fresh media. 25 μl of 5 mg/ml MTT reagent (3-(4,5-dimethylthazol-2-yl)-2,5-diphenyltetrazolium bromide in phosphate-buffered saline, SIGMA, USA) was added to each well of the 96-well plates by multi-channel pipetting, followed by incubation at 37° C. for 4 hours. 150 μl of isopropanol containing 0.1N HCl was added to the cells and incubated at room temperature for 1 hour. Absorbance was measured at 570 nm with Spectramax 190™ (Molecular Devices, USA) to score the amount of cells that survived.

The percentage of growth inhibition was calculated using the following formula:

Percentage of growth inhibition=1−(Absorbance of an experimental well/Absorbance of a control well)×100.

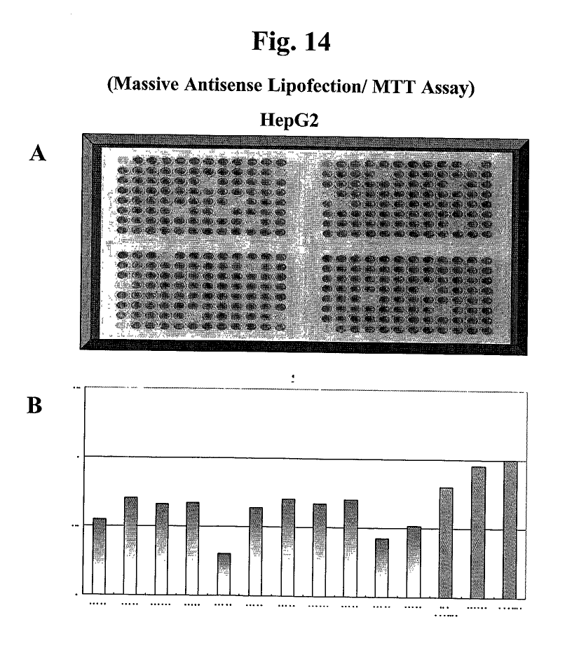

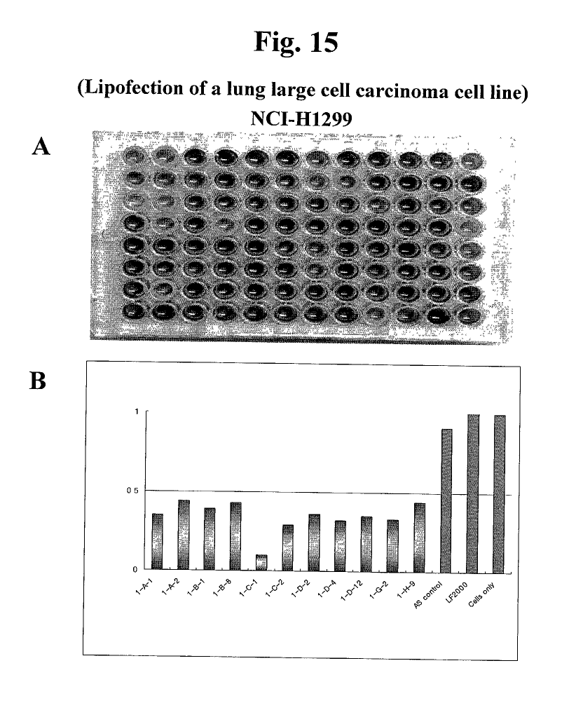

The percentage of growth inhibition in the experimental wells treated with LC-antisense compound-carrier complex, and the control wells that were either sham treated, treated with carrier alone, or were treated with control DNA-carrier complexes, were all measured by optical density, and the recorded absorbance readings compared with each other (FIGS. 14 and 15). Single-stranded DNA without an insert that was purified from bacterial culture was used as control DNA.

From 687 LC-antisense compounds used in the high-throughput assay, 80 antisense compounds (˜12%) in HepG2 (Table 1) and 129 antisense compounds (˜19%) in NCI-H1299 (Table 2) displayed growth-inhibitory effects to varying degrees. Moreover, 22 out of the 80 in HepG2 and 48 out of the 129 genes in NCI-H1299 cell lines were found to have no known function at all. Moreover, genes of unclear function were also found. However, it is clear from the results that these identified genes play a significant role in promoting the proliferation of liver and lung cancer cells.

The cancer cell growth inhibited by LC-antisense compounds was measured first by the above-mentioned MTT reduction assay and confirmed by [³H]-thymidine incorporation assay performed on a configured LC-antisense compound macroarray assembly (FIGS. 16-19). For the [³H]-thymidine incorporation assay, 0.5 μCi of [³H]-thymidine (2.0 Ci/mmol, Amersham Pharmacia Biotech) was added to cells 24 hours after transfection, and the cells were incubated at 37° C. in a CO₂ incubator. After 4 days, the cells were treated with trypsin (Life Technology, USA) and harvested on a glass microfiber filter (GF/C Whatman, Madistone, Kent, UK). The filter was washed with cold phosphate-buffered saline, and then treated with 5% trichloroacetic acid and absolute alcohol, successively. [³H]-thymidine incorporation was measured by a liquid scintillation counter in a mixture solution containing toluene, Triton X-100, 2,5-diphenyloxazole and 1,4-bis[2-(5-phenyloxazoly)]benzene. The percentage of growth inhibition was calculated using the following formula:

Percentage of growth inhibition=1−(cpm of an experimental well/cpm of a control well)×100.

### Example 9

### Functional Profiling of Antisense Compounds Against Disease Cells in a Macroarry Configuration

To obtain an antisense activity profile of the identified genes, cultured cells of Hep3B (liver cancer), NCI-H1299 (non-small lung cancer), AGS (stomach cancer), HT-29 (colon cancer) and HepG2 (liver cancer) were transferred simultaneously to a macroarray assembly constructed of a chosen set of the LC-antisense compounds.

The LC-antisense compounds were mixed with various carriers such as peptides, DOTAP, and cationic liposomes in various ratios (w/w). The mixture was added to various amounts of cells according their growth characteristics. Experimental cells treated with the LC-antisense compound-carrier complex and control cells treated with carrier alone were incubated for 3-5 more days and were subjected to MTT reduction assay twice. To examine the functional profile, the amount of growth inhibition was calculated, and the data were compared between the different types of cancerous cell lines (FIG. 20).

These results demonstrate that not only direct gene functionalization, but validation of target genes for molecular therapeutics to a particular disease can be performed simultaneously with the high throughput system for functional genomics of the present invention.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

TABLE 1
Examples of Functionally Identified Genes Involved in the Growth of
Liver Cancer Cells

| | GenBank |
|---|---|
| | Accession |
| Name of the Gene | Number |
| Homo sapiens, Polymyositis/scleroderma autoantigen | AA458994 |
| Homo sapiens, ESTs | N21972 |
| Homo sapiens, Nuclear matrix protein p84 | NM₀₀₅₁₃₁ |
| Homo sapiens, Gamma-aminobutyric acid(GABA) | M82919 |
| A receptor beta 3 | |
| Homo sapiens, SRY(sex-determining region Y)-box 9 | S74506 |
| Homo sapiens, ESTs | H13112 |
| Homo sapiens, ESTs | AW294133 |
| Homo sapiens, Primase, polypeptide 1 (49 kD) | NM_000946 |
| Homo sapiens, Human EV12 protein gene | M55267 |
| Homo sapiens, epidermal growth factor receptor pathway | AI679737 |
| substrate 8 | |
| Homo sapiens, protein tyrosine phosphatase, | NM_002828 |
| non-receptor type 2 | |

TABLE 2

Examples of Functionally Identified Genes Involved in the Growth of
Lung Cancer Cells

| | GenBank |
|---|---|
| | Accession |
| Name of the Gene | Number |
| Homo sapiens, TGF-β stimulated protein, TSC-22 | AJ222700 |
| Homo sapiens, General transcription factor II H | M95809 |
| Homo sapiens, Cytochrome P450, subfamily IIIA, | AI114634 |
| polypeptide 7 | |
| Homo sapiens, KIAA0094 protein | D42084 |
| Homo sapiens, MAX dimerization protein | NM_002357 |
| Homo sapiens, Serine/treonine kinase 13 | AI564072 |
| (aurora/IPL 1-kike) | |
| Homo sapiens, ESTs | AIO57094 |
| Homo sapiens, Ras-related GTP-binding protein | AI718343 |
| Homo sapiens, MHC class I region ORF | L06175 |
| Homo sapiens, Tumor necrosis factor receptor superfamily, | AA743176 |
| member 7 | |

What is claimed is:

1. A library of a multitude of unique single-stranded nucleic acids, said library comprising:
a multiplicity of compartments, each of said compartments comprising one or more single-stranded LC-antisense compound derived from recombinant bacteriophage or phagemid vector comprising at least one unique unidirectional antisense nucleic acid insert in an aqueous medium,
wherein said LC-antisense compound is capable of being introduced into a host cell, and which is capable of specifically binding to a nucleic acid in said host cell that is substantially complementary to said unique antisense nucleic acid insert.

2. The library of claim 1, wherein the specificity of the unique antisense nucleic acid insert to a target gene is known at the time said library is first made.

3. The library of claim 1, wherein the specificity of a target host cell nucleic acid that controls the expression of a phenotype of the host cell is unknown at the time said library is first made.

4. The library of claim 1, wherein said host cell is a eucaryotic cell.

5. The library of claim 1, wherein each of said compartments contains from about 0.1 μM to about 1 μM of said LC-antisense compound per ml of aqueous medium.

6. The library of claim 1, wherein said bacteriophage or phagemid vector is derived from a filamentous bacteriophage.

7. The library of claim 1, wherein said bacteriophage or phagemid vector comprises bacteriophage or phagemid genomic sequence in which is inserted said unique antisense nucleic acid insert sequence.

8. The library of claim 1, wherein said bacteriophage or phagemid vector comprises more than one kind of unique antisense nucleic acid insert sequence.

9. The library according to claim 1, wherein said multiplicity of compartments comprises a multi-well format of at least 6 wells.

10. The library according to claim 9, wherein said library is configured to be made and used in a substantially automated process.

11. The library according to claim 9, wherein said multiplicity of compartments comprises a multi-well format of at least 96 wells.

12. The library according to claim 1, wherein said host cell is abnormal such that modulation of gene expression is beneficial in returning said host cell to its normal state.

13. The library according to claim 12, wherein said abnormality is cancer, viral infection, immunologic disorders or metabolic diseases.

14. The library according to claim 13, wherein said cancer is liver cancer, lung cancer, stomach cancer, colon cancer, leukemia, cervical cancer, prostate cancer, bladder cancer, pancreatic cancer, skin cancer, ovarian cancer, kidney cancer, or breast cancer.

15. The library according to claim 13, wherein said viral infection is caused by human papilloma virus (HPV), HIV, small pox, mononucleosis (Epstein-Barr virus), hepatitis, or respiratory syncytial virus (RSV).

16. The library according to claim 13, wherein said metabolic disease is phenylketonuria (PKU), primary hypothyroidism, galactosemia, abnormal hemoglobins, types I and II diabetes, or obesity.

17. The library according to claim 13, wherein said immunological disorder is Sjogren's Syndrome, antiphospholipid syndrome, immune complex diseases, Purpura, Schoenlein-Henoch, immunologic deficiency syndromes, systemic lupus erythematosus, immunodeficiency, rheumatism, kidney, or liver sclerosis.

18. A method of making a library comprising a multitude of unique single-stranded nucleic acids, which comprises one or more single-stranded LC-antisense compound derived from recombinant bacteriophage or phagemid vector comprising at least one unique unidirectional antisense nucleic acid insert, comprising:
inserting a nucleic acid fragment unidirectionally into said bacteriophage or phagemid vector by unidirectionally cloning the nucleic acid fragments into said vector.

19. The method according to claim 18, further comprising the step of:
preparing bacterial transformants by introducing the vector containing the insert into competent bacterial cells to make bacterial transformants; and then infecting said transformants with helper phage to produce said single-stranded nucleic acid library.

20. A method for specifically inhibiting growth of liver cancer cells, comprising administering to said cells a large circular antisense compound targeted to polymyositis/scleroderma autoantigen, ESTs (N21972), Nuclear matrix protein p84, Gamma-aminobutyric acid (GABA) A receptor beta 3, SRY (sex-determining region Y)-box 9, ESTs (H13112), ESTs (AW294133), Primase, polypeptide 1 (49 kD), Human EV12 protein gene, epidermal growth factor receptor pathway substrate 8 or protein tyrosine phosphatase, non-receptor type 2.

21. A method for specifically inhibiting growth of lung cancer cells, comprising administering to said cells a large circular antisense compound targeted to TGF-β stimulated protein, TSC-22, General transcription factor II H, Cytochrome P450, subfamily III A, polypeptide 7, KIAA0094 protein (D42084), MAX dimerization protein, Serine/treonine kinase 13 (aurora/IPL 1-kike), ESTs (AI057094), Ras-related GTP-binding protein, MHC class I region ORF, or Tumor necrosis factor receptor superfamily, member 7.

22. A high throughput system for conducting a functional genomics assay with a unigene unidirectional antisense library comprising the steps of:
(i) forming large circular antisense molecule-carrier complexes with said unigene unidirectional antisense library;
(ii) transfecting the complexes into host cells to eliminate endogenously expressed substantially complementary transcripts;
(iii) screening for a change in phenotype of the host cell;
(iv) identifying the gene that caused the change in phenotype in (iii).

23. The high throughput system according to claim 22, which requires further functional testing.

24. The high throughput system according to claim 22, comprising comparing the gene sequence obtained in step (iv) with previously verified clone information to determine homologous genes or the full gene sequence.

25. The high throughput system according to claim 22, wherein the carrier is liposomes, cationic polymers, HVJ-liposomes complexes, peptides or viruses.

26. The high throughput system according to claim 25, wherein the large circular antisense molecule and carrier are mixed in a ratio comprising about 1:3 or about 1:4 w/w.

27. The high throughput system according to claim 22, wherein the assayed phenotype is cell morphology, cell proliferation, cell apoptosis, or cell reaction to a substrate.

28. The high throughtput system according to claim 27, wherein said assay is RT-PCR, Western blot analysis, immunoassay, MTT reduction assay, [³H]-thymidine incorporation assay, colony formation assay, DNA synthesis and chromatin activation, analysis of apoptosis by inspection of cell morphological changes, chromosomal condensation or fragmentation, DNA fragmentation, quantitative assay for apoptosis, signaling mechanisms of apoptosis, activation of cell cycle regulators, complex formation between cell cycle regulators, or assays for changes of metabolic, morphological, physiological and biochemical phenotypes in vitro and in vivo.

29. A high throughput system for conducting massive functional genomics assays, which is performed by applying a unigene unidirectional antisense library to a cell line of a particular disease comprising the following steps:
1) making an antisense library by massively parallel production of LC-antisense compounds to a large number unigenes;
2) plating a population of host cells in multi-well plates;
3) forming an LC-antisense compound-carrier complex with the antisense library of step 1);
4) performing primary gene functional analysis by transfection of the complex of step 3) into the population of host cells; and
5) performing additional functional analysis of the gene screened in step 4).

30. The high throughput system for functional genomics as set forth in claim 29, wherein the unigene LC-antisense compound is prepared by the steps of:
1) preparing a cDNA fragment of a target gene;
2) preparing a recombinant phage or phagemid by inserting the cDNA fragment of step 1) into a phage or phagemid vector that is capable of producing LC-antisense compounds; and
3) producing the LC-antisense compounds containing antisense sequence of the unigene as a part of a single-stranded circular genome made by the recombinant phage or phagemid of step 2).

31. A high throughput system for massive functional genomics performed by applying a macroarray or microarray assembly to disease cells comprising the steps of:
1) making an antisense array by selecting unigene LC-antisense compounds;
2) plating a population of host cells in multi-well plates;
3) forming LC-antisense compound-carrier complexes on the antisense array of step 1);
4) performing primary gene functional analysis by transfection of the complexes of step 3) into the population of cells; and
5) performing additional functional assays of the genes screened in step 4).


SEQUENCE LISTING (9 sequences)

SEQ ID NO: 1 - DNA, 33 bp, Artificial Sequence
  Synthetic Primer
gatcgtcgacgatgagcacagaaagcatgatcc

SEQ ID NO: 2 - DNA, 33 bp, Artificial Sequence
  Synthetic Primer
gatcgaattcgtcacagagcaatgactccaaag

SEQ ID NO: 3 - DNA, 33 bp, Artificial Sequence
  Synthetic Primer
gatcgtcgacgcgccacccggcttcagaatggc

SEQ ID NO: 4 - DNA, 33 bp, Artificial Sequence
  Synthetic Primer
gatcgaattcggtgaagctgccagtgctatccg

SEQ ID NO: 5 - DNA, 25 bp, Artificial Sequence
  Synthetic Primer
cttccagtgccccctcctccaccgc

SEQ ID NO: 6 - DNA, 22 bp, Artificial Sequence
  Synthetic Primer
catctccctccggaaaggacac

SEQ ID NO: 7 - DNA, 20 bp, Artificial Sequence
  Synthetic Primer
cggatgaacacgccagtcgc

SEQ ID NO: 8 - DNA, 22 bp, Artificial Sequence
  Synthetic Primer
gatgagagggagcccatttggg

SEQ ID NO: 9 - DNA, 72 bp, Artificial Sequence
  Synthetic Primer
ccccctcgaggtcgacgatgagcacagaaagcatgatccgagatgtggaactggcagaggaggcgctcccca